SEQUENCE LISTING (8 sequences)

SEQ ID NO: 1 - protein, 25 aa, Artificial Sequence
  Human
KERVGEYSLYIGRHKVTSKVIEKFP

SEQ ID NO: 2 - protein, 12 aa, Artificial Sequence
  Human
ILSAYQGTPLPA

SEQ ID NO: 3 - protein, 11 aa, Artificial Sequence
  Human
IRGYVIIKPLV

SEQ ID NO: 4 - protein, 6 aa, Artificial Sequence
  Human
VFVFPR

SEQ ID NO: 5 - protein, 10 aa, Artificial Sequence
  Human
VGEYSLYIGR

SEQ ID NO: 6 - protein, 12 aa, Artificial Sequence
  Human
AYSLFSYNTQGR

SEQ ID NO: 7 - protein, 10 aa, Artificial Sequence
  Human
QGYFVEAQPK

SEQ ID NO: 8 - protein, 13 aa, Artificial Sequence
  Human
IVLGQEQDSYGGK

(19) United States
(12) Patent Application Publication
(10) Pub. No.: US 2010/0297074 A1
(43) Pub. Date: Nov. 25, 2010

(54) Wound healing compositions, systems, and methods

(76) Inventors: Richard Hans Gomer, College Station, TX (US); Darrell Pilling, Pearland, TX (US)

Correspondence Address: BAKER BOTTS L.L.P.;PATENT DEPARTMENT, 98 SAN JACINTO BLVD., SUITE 1500, AUSTIN, TX, 78701-4039, US

(21) Appl. No.: 12/798,729

(22) Filed: Apr. 9, 2010

Related U.S. Application Data
(63) Continuation-in-part of application No. 11/158,723, filed on Jun. 22, 2005.
Application No. 11/158,723 is a continuation-in-part of application No. PCT/US03/41183, filed on Dec. 22, 2003.
(60) Provisional application No. 60/436,046, filed on Dec. 23, 2002.
(60) Provisional application No. 60/436,027, filed on Dec. 23, 2002.
(60) Provisional application No. 60/515,776, filed on Oct. 30, 2003.
(60) Provisional application No. 60/519,467, filed on Nov. 12, 2003.
(60) Provisional application No. 60/525,175, filed on Nov. 26, 2003.

(51) Int. Cl. A61K 38/20; A61K 31/729; A61K 38/18; A61P 17/02

(52) U.S. Cl. 424/85.2; 514/54; 514/9.1; 514/8.9

(57) ABSTRACT

The present disclosure relates, according to some embodiments, to the ability of SAP to suppress the differentiation of monocytes into fibrocytes. It also relates to the ability of IL-4 and IL-3 to enhance the differentiation of monocytes into fibrocytes. Methods and compositions for binding SAP, decreasing SAP levels and suppressing SAP activity are provided. Methods of using, inter alia, CPHPC, the 4,6-pyruvate acetyl of beta-D-galactopyranose, ethanolamines, high EEO agarose, IL-4, and IL-13, and anti-SAP antibodies and fragments thereof to increase monocyte differentiation into fibrocytes are provided. These methods are useful in a variety of applications, including wound healing. Wound dressings are also provided. Finally, the disclosure may include assays for detecting the ability of various agents to modulate monocyte differentiation into fibrocytes and to detect monocyte defects.

### PRIORITY CLAIM

The present application claims priority as a continuation-in-part under 35 U.S.C. §120 to U.S. patent application Ser. No. 11/158,723, filed Jun. 22, 2005, now U.S. Pat. No. ______, which is a continuation-in-part under 35 U.S.C. §120 of PCT/US03/41183, filed Dec. 23, 2003 in English designating the U.S., which claims priority to U.S. Provisional Patent Application Ser. No. 60/525,175, filed Nov. 26, 2003; U.S. Provisional Patent Application Ser. No. 60/519,467, filed Nov. 22, 2003; U.S. Provisional Patent Application Ser. No. 60/515,776, filed Oct. 30, 2003; U.S. Provisional Patent Application Ser. No. 60/436,027, filed Dec. 23, 2002; and U.S. Provisional Patent Application Ser. No. 60/436,046, filed Dec. 23, 2002, all of which are incorporated by reference herein.

### FIELD OF THE INVENTION

The present disclosure relates, in some embodiments, to wound healing compositions, systems, and methods.

### BACKGROUND OF THE DISCLOSURE

### Fibrocytes

Inflammation is the coordinated response to tissue injury or infection. The initiating events are mediated by local release of chemotactic factors, platelet activation, and initiation of the coagulation and complement pathways. These events stimulate the local endothelium, promoting the extravasation of neutrophils and monocytes. The second phase of inflammation is characterized by the influx into the tissue of cells of the adaptive immune system, including lymphocytes. The subsequent resolution phase, when apoptosis of the excess leukocytes and engulfment by tissue macrophages takes place, is also characterized by repair of tissue damage by stromal cells, such as fibroblasts.

Both IL-4 and IL-13 are potent activators of the fibrotic response. Fibrosis is the excess production of connective tissue, especially collagen, following tissue damage or inflammation. IL-4 is known to enhance wound repair and healing. IL-13 and IL-4 in many systems act in a similar manner. However, key differences have been found in the function of these two proteins in various circumstances. For instance, IL-13 is more dominant in resisting infection by intestinal nematodes and intracellular parasites, such as Leishmania. IL-13 also plays a much more significant role than IL-4 in asthma. In contrast, IL-4 is more dominant than IL-13 in stimulating B cell production of immunoglobulin and in T cell survival and differentiation.

TGFβ, which is also known to play a role in wound healing, had been shown to facilitate fibrocyte differentiation into myofibroblasts, which are further associated with wound healing.

There appear to be multiple sources of fibroblast-like cells responsible for repair of wound lesions or in other fibrotic responses. Local quiescent fibroblasts migrate into the affected area, produce extracellular matrix proteins, and promote wound contraction or fibrosis. In addition, circulating cells (called fibrocyte precursors) present within the blood migrate to the sites of injury or fibrosis, where they can differentiate into fibroblast-like cells called fibrocytes and mediate tissue repair and other fibrotic responses.

Fibrocytes are known to differentiate from a CD14+ peripheral blood monocyte precursor population. Fibrocytes express markers of both hematopoietic cells (CD45, MHC class II, CD34) and stromal cells (collagen types I and III and fibronectin). Mature fibrocytes rapidly enter sites of tissue injury where they secrete inflammatory cytokines. Once there, fibrocytes can function as antigen presenting cells (APCs), thereby inducing antigen-specific immunity. Fibrocytes are also capable of secreting extracellular matrix proteins, cytokines and pro-angiogenic molecules, which may aid in wound repair.

Fibrocytes are also associated with a variety of other processes and disorders. They are associated with the formation of fibrotic lesions after Schistosoma japonicum infection in mice and are also implicated in fibrosis associated with autoimmune diseases. Fibrocytes have also been implicated in pathogenic fibrosis such as that associated with radiation damage, Lyme disease and pulmonary fibrosis. CD34+ fibrocytes have also been associated with stromal remodeling in pancreatitis and stromal fibrosis. Finally, fibrocytes have been shown to promote angiogenesis by acting on endothelial cells.

### Serum Amyloid P

Serum amyloid P (SAP), a member of the pentraxin family of proteins that include C-reactive protein (CRP), is secreted by the liver and circulates in the blood as stable pentamers. The exact biological role of SAP is still unclear. SAP binds to sugar residues on the surface of bacteria leading to their opsonisation and engulfment. SAP also binds to free DNA and chromatin generated by apoptotic cells at the resolution of an immune response, thus preventing a secondary inflammatory response. Molecules bound by SAP are removed from extracellular areas due to the ability of SAP to bind to all three classical Fcγ receptors (FcγR). After receptor binding, SAP and any attached molecule are likely engulfed by the cell.

FcγR are necessary for the binding of IgG to a wide variety of hematopoietic cells. Peripheral blood monocytes express both CD64 (FcγRI) and CD32(FcγRII), whereas tissue macrophages express all three classical FcγR. A subpopulation of monocytes also express CD16 (FcγRIII).

Clustering of FcγR on monocytes by IgG, either bound to pathogens or as part of an immune complex, initiates a wide variety of biochemical events. The initial events following receptor aggregation include the activation of a series of src kinase proteins. In monocytes, these include lyn, hck and fgr, which phosphorylate tyrosine residues on the ITAM motif of the FcR-γ chain associated with FcγRI and FcγRIII, or the ITAM motif with the cytoplasmic domain of FcγRIIa. Phosphorylated ITAMs lead to the binding of a second set of src kinases, including syk. Syk has been shown to be vital for phagocytosis of IgG-coated particles. However, the wide distribution of syk in non-hematopoietic cells and the evidence that syk is involved in both integrin and G-protein coupled receptor signaling, indicates that this molecule has many functions.

Both SAP and CRP augment phagocytosis and bind to Fcγ receptors on a variety of cells. CRP binds with a high affinity to FcγRII (CD32), a lower affinity to FcγRI (CD64), but does not bind FcγRIII (CD 16). SAP binds to all three classical Fcγ receptors, with a preference for FcγRI and FcγRII, particularly FCγRI. Although there are conflicting observations on the binding of CRP to FcγR, both SAP and CRP have been shown to bind to Fc receptors and initiate intracellular signaling events consistent with FcγR ligation.

In human blood serum, males normally have approximately 32 μg/ml +/−7 μg/ml of SAP, with a range of 12-50 μg/ml being normal. Human females generally have approximately 24 μg/ml +/−8 μg/ml of SAP in blood serum, with a range of 8-55 μg/ml being normal. In human cerebral spinal fluid there is normally approximately 12.8 ng/ml SAP in human males and approximately 8.5 ng/ml in females. Combining male and female data, the normal SAP level in human serum is 26 μg/ml +/−8 μg/ml with a range of 12-55 μg/ml being normal. (The above serum levels are expressed as mean +/− standard deviation.)

SAP has been investigated primarily in relation to its role in amyloidosis. Recently, a drug, R-1-[6-[R-2-carboxy-pyrrolidin-1-yl]-6-oxo-hexanoyl] pyrrolidine-2-carboxylic acid (CPHPC) was developed to deplete SAP and thereby treat amyloidosis. However, this drug appears to have been applied systemically and not to have been used to treat wound healing or to have other localized or systemic effects.

Agar has been previously used as a wound dressing. However, it is not clear whether such previous wound dressings were capable of depleting SAP because they may not have contained appropriate chemical moieties or may have been used inappropriately. In any event, these previous wound dressing do not appear to have incorporated any additional wound healing factors. Further the dressings appear to have been used only for external wounds. Finally, it does not appear that these dressings incorporated purified SAP depleting chemicals or enhanced levels thereof.

### SUMMARY

Accordingly, a need has arisen for improved compositions, systems, and methods for promoting wound healing. The present disclosure relates, in some embodiments, to wound healing compositions, systems, and methods.

The present disclosure may include compositions and methods for binding SAP. Compositions operable to bind SAP may include CPHPC, the 4,6-pyruvate acetyl of beta-D-galactopyranose, phosphoethanolamines, and anti-SAP antibodies or fragments thereof. Such binding may occur in vivo.

The disclosure may also include compositions and methods for the depletion of SAP levels in a sample. The sample may be located in vitro or in vivo. In vivo the sample may include an entire organism or a portion thereof and depletion may be systemic or may be confined to a particular area, such as an organ or wound. The compositions may include those supplied directly or produced in the sample, for instance through expression of a transgene. Compositions operable to deplete SAP may include CPHPC, high EEO agarose, the 4,6-pyruvate acetyl of beta-D-galactopyranose, phosphoethanolamine, and anti-SAP antibodies or fragments thereof. SAP levels in a sample may also be depleted by interfering with its initial production or increasing degradation.

The disclosure may also include compositions and methods for the suppression of SAP activity. Suppression may be in a sample and may occur in vitro or in vivo. Compositions also include compositions supplied directly to a sample and those produced in the sample, such as by expression of a transgene. These compositions may act by decreasing SAP formation, decreasing the ability of SAP proteins to interact with monocytes or tissue macrophages, decreasing the ability of SAP proteins to interact with cofactors or decreasing the level of such cofactors, and interfering with SAP-induced signaling in monocytes, such as a pathway triggered by SAP binding to an FcγR. Compositions operable to suppress SAP activity may include anti-SAP antibodies and fragments thereof, particularly those targeted the Fc-binding region.

The disclosure may additionally include methods and compositions for promoting wound healing by depleting or suppressing SAP in the region of a wound. Compositions may also include additional wound healing factors. In specific embodiments of the disclosure, wound healing compositions may include high EEO agarose, phosphoethanolamine agarose, Ca²⁺, and combinations thereof. Cytokines such as IL-13, IL-4 and TGFβ may be added to these compositions.

Yet another aspect of the disclosure relates to compositions and methods for promoting fibrocyte formation by providing IL-4, IL-13 or a combination of the two to monocytes. The monocytes may be located in vitro or in vivo. IL-4 and IL-13 may be provided by an extraneous source, or endogenous production may be increased.

Finally, the disclosure may include assays to detect the ability of a sample to modulate fibrocyte differentiation from monocytes. In one embodiment, normal monocytes may be supplied with the sample. The sample may include normal SAP. It may also include SAP or a biological fluid from a patient such as a patient with a wound healing disorder, or it may include a potential drug. In another embodiment, the sample may include normal SAP while the monocytes may be derived from a patient and may be abnormal. In either type of assay, the effects on monocyte differentiation into fibrocytes may be compared with a normal control to detect any increases or decreases in monocyte differentiation as compared to normal.

### BRIEF DESCRIPTION OF THE DRAWINGS

FIG. 1 illustrates the effects of serum and plasma on the rapid differentiation of fibroblast-like cells. In FIG. 1A peripheral blood mononuclear cells (PBMC) at 2.5×10⁵ per ml were cultured in serum-free medium for 3 or 6 days in the presence or absence of 0.1% human serum and then examined by microscopy for the appearance of fibroblast-like cells. Bar is 100 μm.

In FIG. 1B PBMC at 2.5×10⁵ per ml were cultured in serum-free medium for 6 days in dilutions of human plasma. Cells were then air-dried, fixed, stained, and fibrocytes were enumerated by morphology. Results are expressed as mean ±SD of the number of fibrocytes per 2.5×10⁵ PBMCs (n=5 experiments). Stars indicate values that are statistically significant differences from the cells cultured in the absence of SAP.

FIG. 2 illustrates the expression of surface molecules on fibroblast-like cells. PBMC were cultured on glass slides in serum-free medium for 6 days. Cells were air-dried and analyzed by immunohistochemistry. Monoclonal antibodies used were as indicated, and identified by biotin-conjugated goat anti-mouse Ig followed by ExtrAvidin peroxidase. Cells were counterstained with Mayer's haematoxylin to identify nuclei. Positive staining was identified by brown staining, nuclei were counterstained blue. An insert for CD83 was used to indicate positive staining on a dendritic cell.

FIG. 3 illustrates the characterization of the molecule present in plasma that inhibits fibrocyte differentiation. Citrated plasma was treated with BaCl₂ and the precipitated material was collected by centrifugation and dialyzed against 10 mM sodium phosphate containing 10 mM EDTA and protease inhibitors. This material was then fractionated by heparin and ion exchange chromatography.

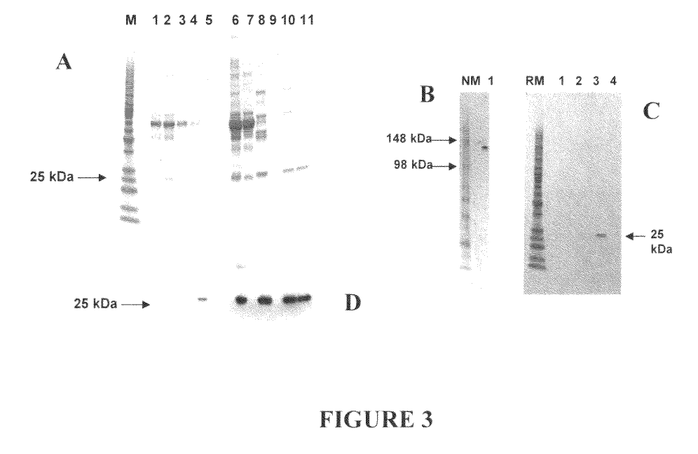

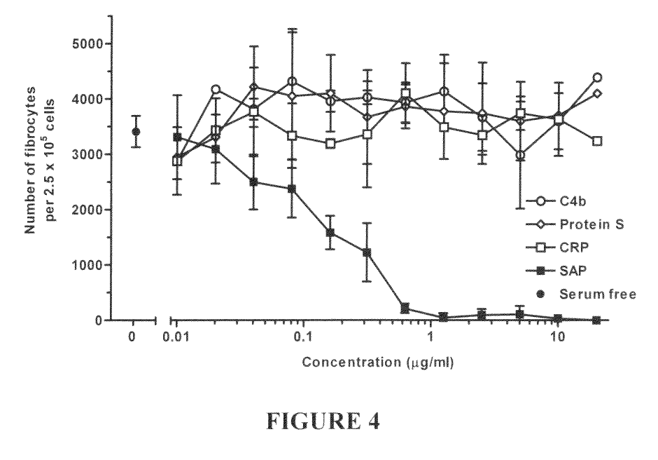

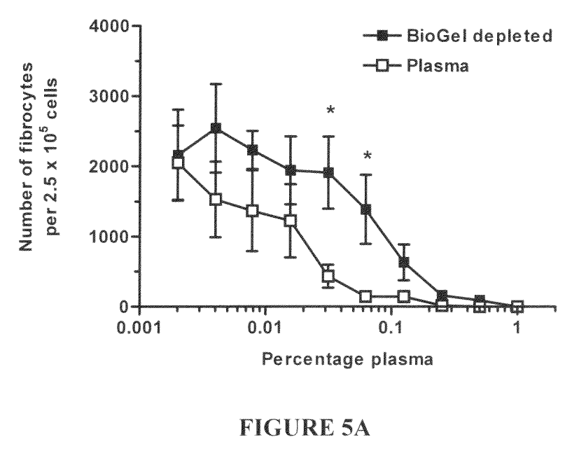

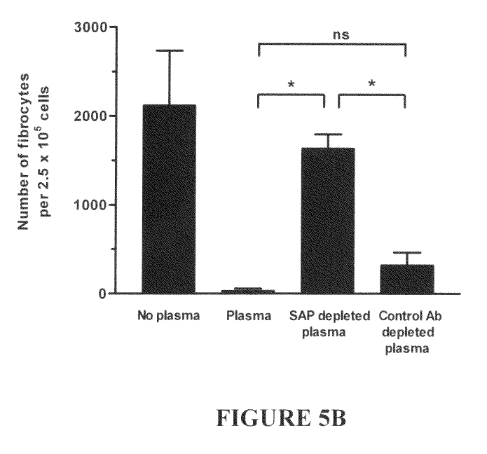

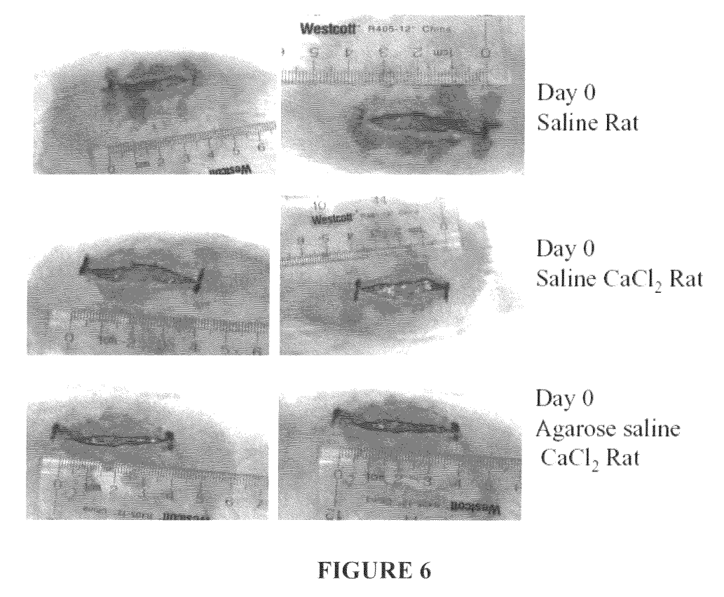

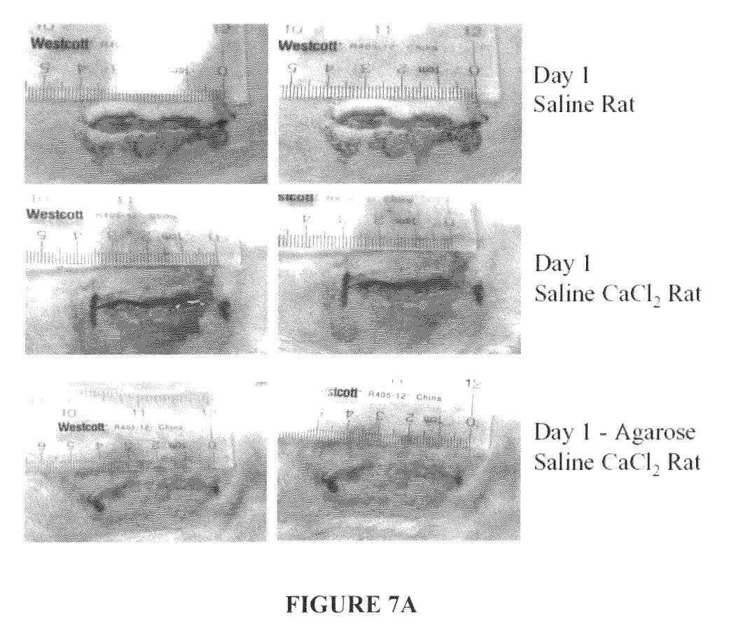

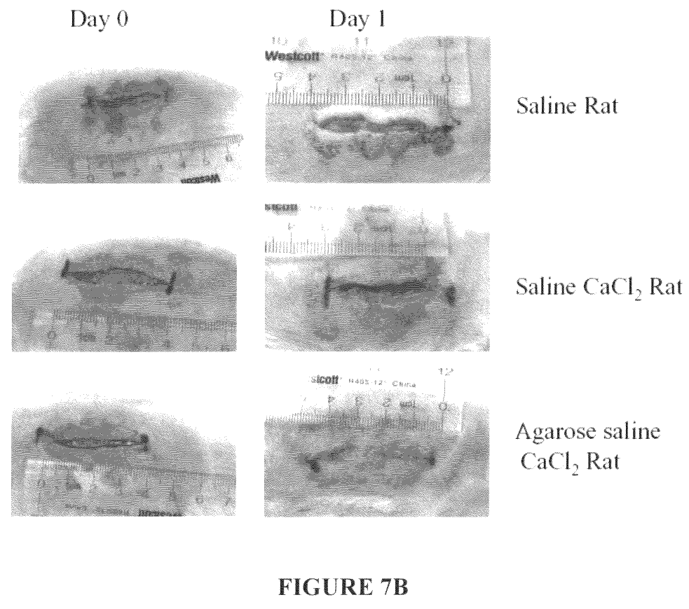

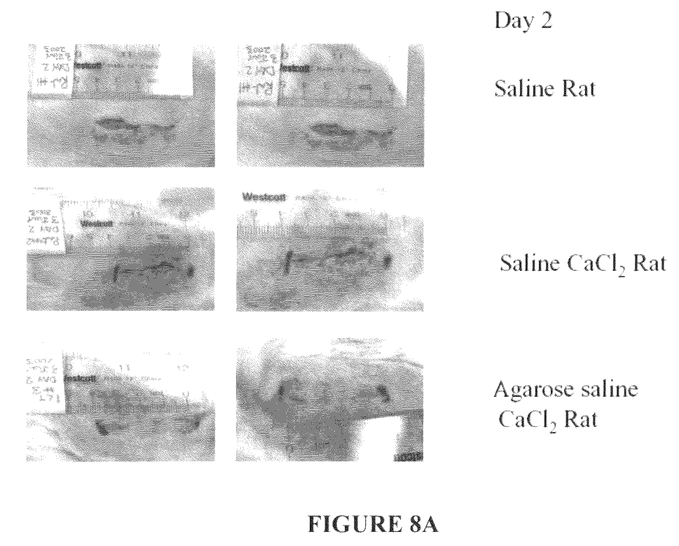

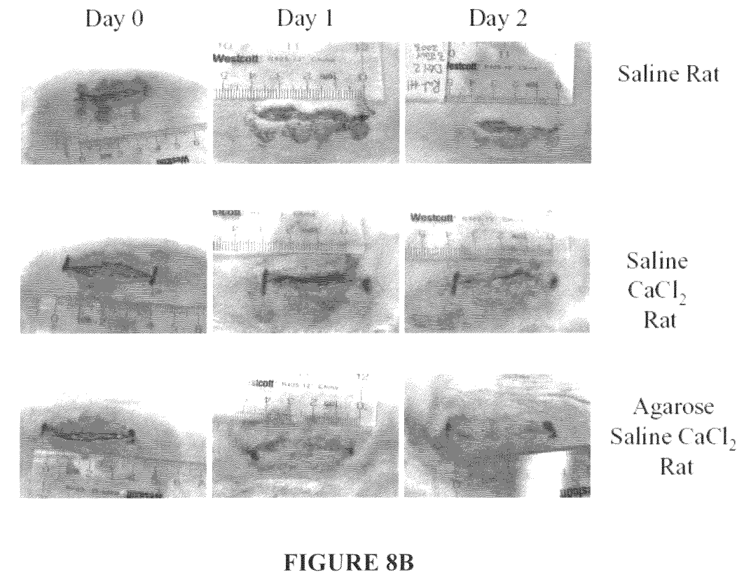

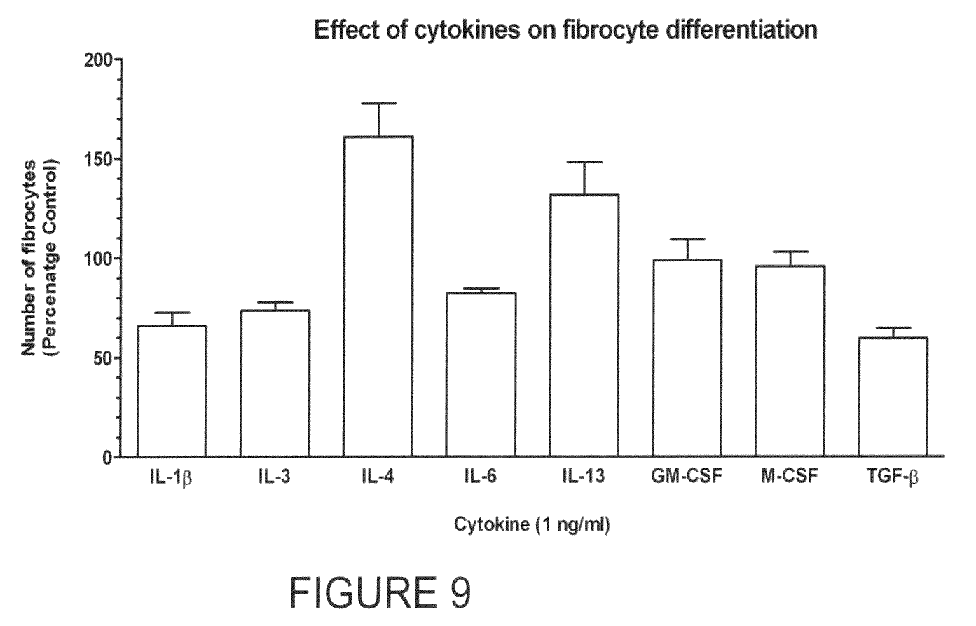

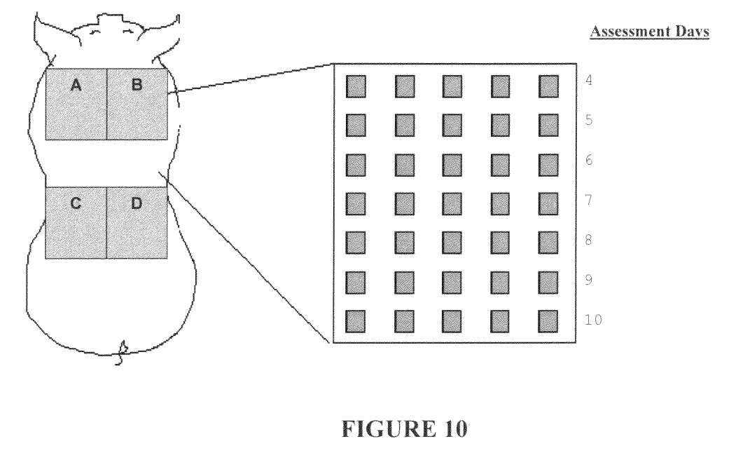

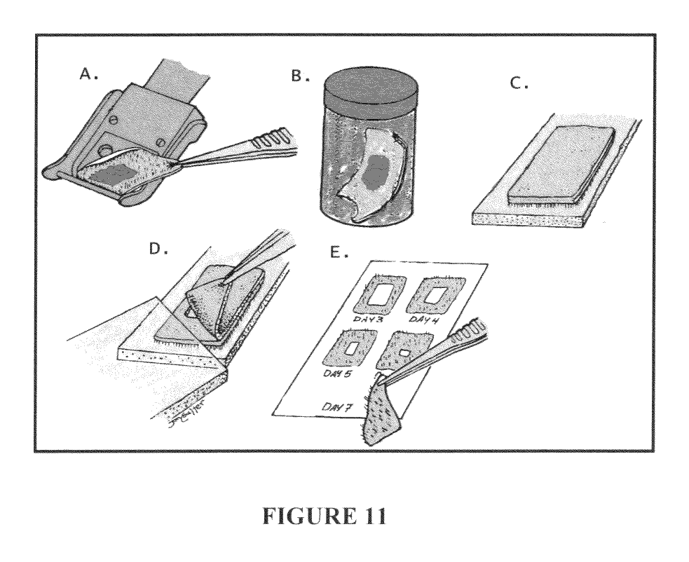

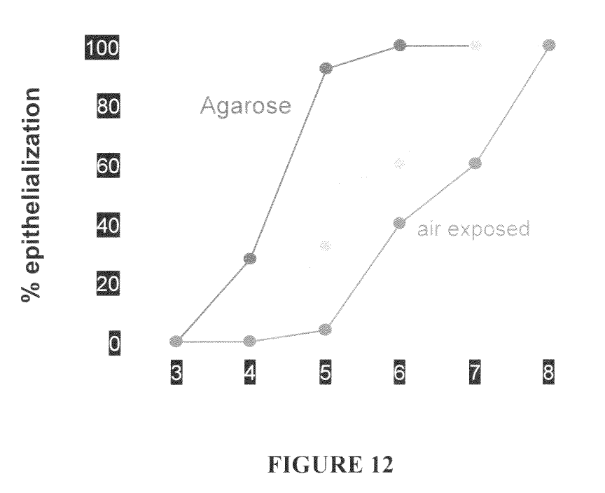

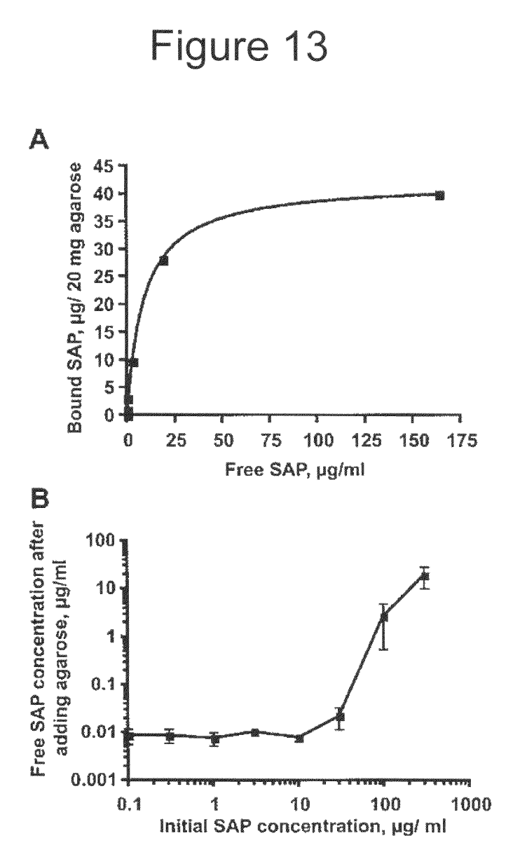

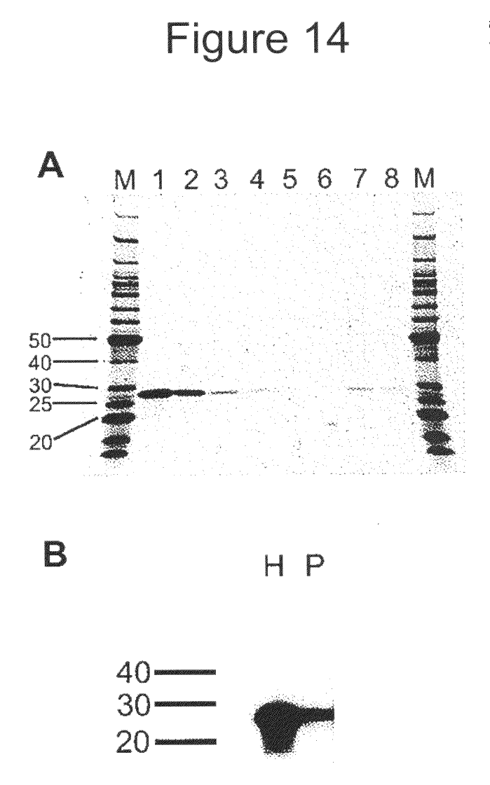

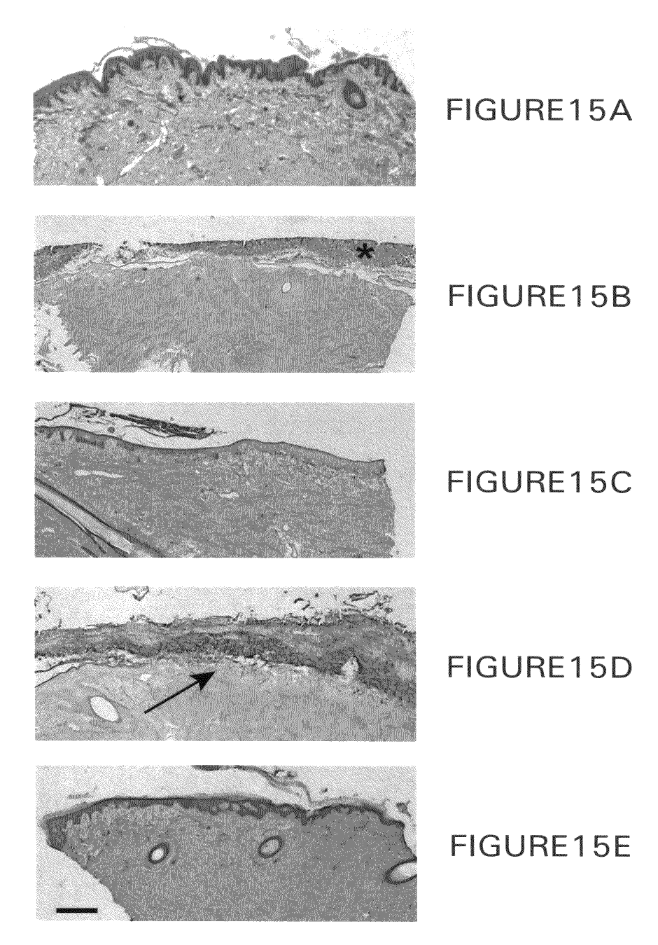

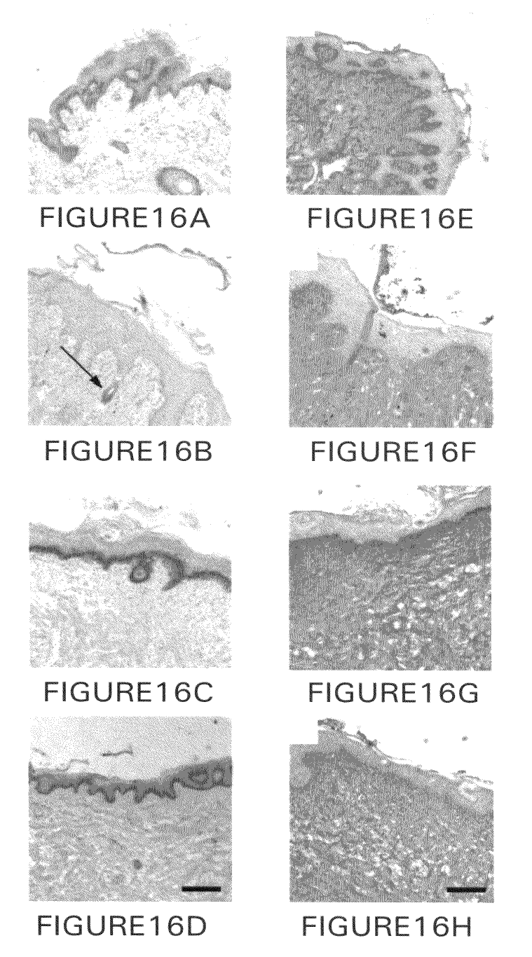

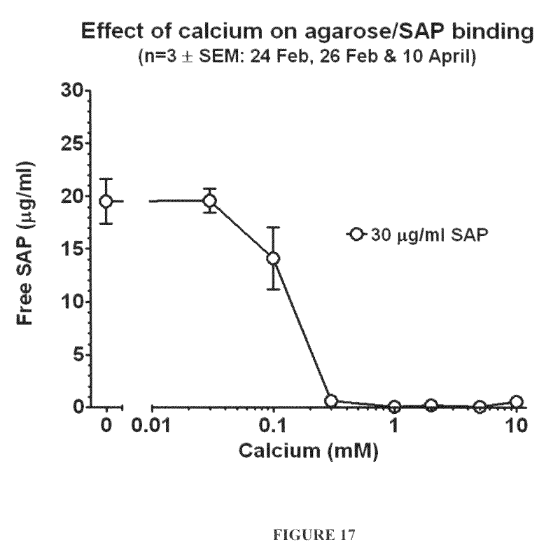

In FIG. 3A fractions were analyzed by PAGE on a 4-20% reducing gel and stained with coomassie blue. M indicates molecular weight markers. Lane 1 contained plasma, lane 2 contained BaCl₂ supernatant, lane 3 contained wash 1, lane 4 contained wash 2, lane 5 contained BaCl₂ precipitate, lane 6 contained BaCl₂ precipitate, lane 7 contained heparin flow through, lane 8 contained the heparin fraction, lane 9 contained High Q flow through, lane contained the 10 High Q fraction, lane 11 contained the gel purified fraction. Lanes 1-5 diluted 1:500 in sodium phosphate buffer, lanes 6-11 undiluted.

Active fractions eluted off the High Q ion exchange column and gel slices were analyzed by 4-20% PAGE on a native gel in FIG. 3B and a reducing gel in FIG. 3C. NM indicates native gel markers, RM indicates reduced gel markers, in FIG. 3B lanes 1-3 are control gel samples, lane 4 contained active fraction. In FIG. 3D fractions were assessed by western blotting, using a rabbit anti-SAP antibody. Lanes 1-11 correspond to those in FIG. 3A.

FIG. 4 shows the inhibition of fibrocyte formation by SAP, but not CRP or other plasma proteins. PBMC at 2.5×10⁵ per ml were cultured in serum-free medium for 6 days in the presence of commercially available purified SAP (filled square), CRP (open square), Protein S (open diamond)or C4b (open circle) and then examined for the appearance of fibroblast-like cells. Cells were then air-dried, fixed, stained and fibrocytes enumerated by morphology. Results are mean ±SD of fibrocytes per 2.5×10⁵ PBMC (n=3 separate experiments).

FIG. 5 shows the effect of depletion of SAP from plasma in a fibrocyte differentiation assay.

FIG. 5A shows the effect on fibrocyte differentiation of depleting SAP from plasma with BioGel agarose beads. Number of fibrocytes found in an assay supplied with either plasma (open square) or BioGel depleted plasma (filled square) at a variety of dilutions is shown.

FIG. 5B shows the number of fibrocytes formed in an assay performed with no plasma or equal dilutions of plasma, anti-SAP antibody depleted plasma or control (irrelevant) antibody depleted plasma. Stars indicate statistically significant differences.

FIG. 6 shows initial skin incisions on three different rats to be treated with saline, saline with CaCl₂, or agarose with saline and CaCl₂.

FIG. 7 shows healing of the skin incisions shown in FIG. 6. FIG. 7A shows healing of skin incisions on three different rats after one day of treatment with either saline, saline with CaCl₂, or agarose with saline and CaCl₂. FIG. 7B shows a comparison of initial skin incisions on three different rats and healing after one day of treatment with either saline, saline with CaCl₂, or agarose with saline and CaCl₂.

FIG. 8 also shows healing of skin incisions on rats. FIG. 8A shows healing of skin incisions on three different rats after two days of treatment with either saline, saline with CaCl₂, or agarose with saline and CaCl₂. FIG. 8B shows a comparison of initial skin incisions on three different rats and healing after one and two days of treatment with either saline, saline with CaCl₂, or agarose with saline and CaCl₂.

FIG. 9 shows the effects of various cytokines on promotion of fibrocyte differentiation.

FIG. 10 shows an experimental setup in a porcine model.

FIG. 11 shows the steps of an epidermal migration assessment in a porcine model. A: wound excision; B: placement of specimen in sodium bromide for incubation; C: placement of specimen on glass slide for separation; D: separation of specimen; D: placement of epidermal specimen on cardboard for permanent record.

FIG. 12 shows the combined healing data from porcine wound healing studies. Day after wounding is indicated on the x axis.

FIG. 13A shows the binding of human SAP to SP agarose. Binding data were plotted and a one-site binding model (solid line) was fitted to the data.

FIG. 13B shows the binding of human SAP to SP agarose, plotting the SAP concentration before and after adding a 1:5 w/v ratio of agarose beads to the SAP solution. Values are mean ±SEM (n=3). The absence of an error bar indicates that the error was smaller than the plot symbol.

FIG. 14A shows the specificity of the high-affinity binding of human and porcine serum proteins to SP agarose. Human and porcine sera were incubated with SP agarose, washed, and bound material was eluted and separated on an SDS-polyacrylamide gel which was then stained with Coomassie. Lanes are M, molecular mass markers (molecular masses in kDa are indicated at left); 1, 1 μg human SAP; 2, 0.3 μg human SAP; 3, 0.1 μg human SAP; 4, 0.03 μg human SAP; 5, 0.01 μg human SAP; 6, 0.003 μg human SAP; 7, 10 μg of the eluted material from human serum, 8, 10 μl of the eluted material from porcine serum.

FIG. 14B shows the specificity of the high-affinity binding of human and porcine serum proteins to SP agarose. A Western blot of the protein eluted from SP agarose was stained with anti-human SAP antibodies. H is the material from human serum; P is the material from porcine serum. The position of molecular mass markers (in kDa) is indicated at left.

FIGS. 15A-15E show sections of wounds stained with hematoxylin and eosin.

FIGS. 15A-15E are at the same scale, and the bar in E is 0.5 mm.

FIG. 15A shows a section of skin before wounding.

FIG. 15B shows an untreated wound at day 4. The * shows an area of crust.

FIG. 15C shows an agarose in carbomer-treated wound at day 4.

FIG. 15D shows an untreated wound at day 7. The arrow shows a region of epidermis under the crust.

FIG. 15E shows an agarose in carbomer-treated wound at day 7.

FIGS. 16A-16H show the detection of cytokeratin and collagen-I as described below. In each FIG. 16A-16H, the bar shown is 0.2 mm.

FIG. 16A shows the detection of cytokeratin. Cryosections were stained with anti-cytokeratin antibodies. Normal skin is shown.

FIG. 16B shows the detection of cytokeratin in day 10 wounds. Cryosections were stained with anti-cytokeratin antibodies to show re-epithelialization. A day 10 untreated wound is shown (the arrow shows a hair follicle).

FIG. 16C shows the detection of cytokeratin in day 10 wounds. Cryosections were stained with anti-cytokeratin antibodies to show re-epithelialization. An agarose in carbomer-treated wound is shown.

FIG. 16D shows the detection of cytokeratin in day 10 wounds. Cryosections were stained with anti-cytokeratin antibodies to show re-epithelialization. An IntraSite hydrogel-treated wound is shown.

FIG. 16E shows the detection of collagen-I. Sections were stained with anti-collagen-I antibodies to show dermal remodeling. Normal skin is shown.

FIG. 16F shows the detection of collagen-I in day 10 wounds. Sections were stained with anti-collagen-I antibodies to show dermal remodeling. A day 10 untreated wounds is shown.

FIG. 16G shows the detection of collagen-I in day 10 wounds. Sections were stained with anti-collagen-I antibodies to show dermal remodeling. A wound treated with agarose in carbomer is shown.

FIG. 16H shows the detection of collagen-I in day 10 wounds. Sections were stained with anti-collagen-I antibodies to show dermal remodeling. A wound treated with IntraSite hydrogel is shown.

FIG. 17 shows the effect of calcium concentration on agarose binding SAP. SP Sepharose FF beads were incubated for 60 minutes with 30 micro g/ml SAP in 10 mM Tris pH 8.0/140 mM NaCl, with increasing concentrations of calcium (calcium chloride). Supernatants were then collected and assayed for SAP by ELISA. The graph show the amount of SAP remaining in the supernatant. Results are expressed as mean ±SEM (n=3 experiments). The absence of error bars indicates that the error was smaller than the plot symbol.

### DETAILED DESCRIPTION

The present disclosure relates, in some embodiments, to wound healing compositions, systems, and methods. In some embodiments, the present disclosure relates to the ability of SAP to suppress the differentiation of monocytes into fibrocytes. Accordingly, some embodiments of the disclosure may include compositions, systems, and methods for increasing such differentiation. These compositions and methods may be useful in a variety of applications in which increased fibrocyte formation is beneficial, such as wound healing. The disclosure may additionally include methods for detecting problems in the ability of monocytes to differentiate into fibrocytes or for SAP to inhibit this differentiation. These problems may be correlated with a disease or may be drug-induced.

Fibrocytes are a distinct population of fibroblast-like cells derived from peripheral blood monocytes. Culturing CD 14+ peripheral blood monocytes in the absence of serum or plasma leads to the rapid differentiation of fibrocytes. This process normally occurs within 48-72 hours and is suppressed by the presence of blood serum or plasma. Experiments described further herein have determined that this suppression is caused by SAP. Additional experiments have determined that, when monocytes are cultured in serum-free medium, differentiation into fibrocytes is enhanced by the presence of IL-4 or IL-13.

### Binding of SAP

The present disclosure may include compositions and methods for binding SAP. Compositions may include CPHPC, the 4,6-pyruvate acetyl of beta-D-galactopyranose, ethanolamines, anti-SAP antibodies or fragments thereof, and DNA. Agarose may also be used to bind SAP. For example, High EEO agarose (Fisher Scientific International Inc., N.H.), Low EEO agarose (Fisher Scientific International Inc., N.H.), SeaKem® ME agarose (Cambrex Bioscience, N.J.), SeaKem® SP agarose (Cambrex Bioscience, N.J.), Bio-Gel A (BioRad Laboratories, Calif.), SP-Sepharose (Amersham Biosciences, UK) CL-Sepharose (Amersham Biosciences, UK), Heparin-agarose, Aspartic acid-agarose and Poly-lysine-agarose and derivatized agarose may all be used in embodiments of the disclosure. Binding to a pyruvate acetyl may play a significant role in SAP binding to agarose.

These compositions may include purified chemicals, or the chemicals may be attached to another compound, for example a much larger compound, such as agarose or a biocompatible polymer (e.g. PEG, poly(amino acids) such as poly(glutamic acid), chitosan, other polysaccharides, and other biological polymers, or chemically modified versions thereof).

SAP may also bind to a variety of other things. For example, it may bind to cells and tissues. SAP may bind to monocytes in a non-calcium dependent manner. This binding may be inhibited by C1q. SAP may bind to the basement membranes of blood vessels and renal tissues.

SAP may bind to proteins, such as elastin. SAP may bind to collagen-IV at around 10⁷ or 10⁸ M in a calcium-dependent manner inhibited by C1q or high levels of CRP, but not phosphoethanolamime. SAP may bind to laminin at around 3×10⁷ M in a calcium-dependent manner inhibited by CRP and phosphatidylethanolamine. SAP may bind fibronectin in a calcium-dependent manner. SAP may also bind to amyloid deposits in a calcium-dependent manner. SAP may bind to keyhole limpet haemocyanin (KLH)-conjugated macromolecules. SAP may also bind to C1q and C4bp. SAP may bind to sphingomyelinase D. SAP may also bind to many proteins with terminal mannose residues, such as ovalbumin, thyroglobulin, beta-glucuronidase and C3bi.

SAP may bind receptors, such as L-selectin (CD62-L) in a calcium-dependent manner. This binding appears to be due to N-linked carbohydrate domains on the L-selectin. SAP also binds to Fc receptors.

SAP may bind bacteria including Mycobacterium tuberculosis, Streptococcus pneumoniae, Klebsiella rhinoscleromatis, group A Streptococcus pyogenes, Neisseria meningitidis, including a lipopolysaccharide (LPS)-negative mutant, and rough variants of Escherichia coli. SAP also binds to bacterial lipopolysaccharide (LPS).

SAP may bind to Influenza A and may even interfere with infection by that virus.

SAP may also bind to various bodily and cellular debris. For example, SAP may bind to many aggregated or immobilized proteins, such as aggregated IgG. SAP may also bind to apoptotic cells, histones, chromatin, and DNA.

SAP may bind to a variety of carbohydrates in addition to those already discussed. For example SAP may bind to 6-phosphorylated mannose and the sulphated saccharides galactose, N-acetyl-galactosamine and glucuronic acid, to heparin, dermatan sulphate, and chondroitin sulfate. SAP my bind to zymosan in a calcium-dependent manner.

SAP may bind to lipids such as phospholipids, oxidized LDL, and colocalises with apolipoprotein A-I (apoA-I), apoB, apoC-II, and apoE in human coronary arteries.

SAP may further bind to a variety of plastic materials such as polypropylene, polyethylene terephthalate (PET), or polydimethylsiloxane (PDMS) in a calcium dependent manner.

SAP may also bind to pectic acid, and trinitrophenol (TNP)-conjugated macromolecules.

Binding may occur in vitro or in vivo. Binding to one or more the above items may be used to deplete SAP from a wound or may be taken into account in wound healing and wound treatment.

According to some embodiments, SAP may be bound by a composition including approximately 1% w/v high EEO agarose. The composition may also include a cation, such as Mg²⁺ or Ca²⁺. For example, the agarose may include from approximately 2 mM CaCl₂ to approximately 5 mM CaCl₂. According to some embodiments, a wound healing composition may comprise an agarose and a divalent cation. The divalent cation may be present at a concentration of up to approximately 2 mM, up to approximately 5 mM, from approximately 2 mM to approximately 5 mM, over approximately 2 mM, over approximately 5 mM, and/or combinations thereof. In one example, the divalent cation may be present at a concentration of approximately 0.3 mM.

In other embodiments, the composition may include an antibody or antibody fragment that targets the portion of SAP functional in inhibiting fibrocyte formation from monocytes. In an exemplary embodiment, the functional portion of SAP may be selected from the region that does not share sequence homology with CRP, which has no effect on fibrocyte formation. For instance amino acids 65-89 KERVGEYSLYIGRHKVTSKVIEKFP (SEQ.ID.NO.1) of SAP are not homologous to CRP. Amino acids 170-181 ILSAYQGTPLPA (SEQ.ID.NO.2) and 192-205 IRGYVIIKPLV (SEQ.ID.NO.3) are also not homologous. Additionally a number of single amino acid differences between the two proteins are known and may result in functional differences.

### Depletion of SAP

Other aspects of the disclosure relate to compositions and methods for the depletion of SAP levels in a sample. The sample may be located in vitro or in vivo. In vitro samples may include tissue cultures, bioreactors, tissue engineering scaffolds and biopsies. In vivo the sample may include an entire organism or a portion thereof such as an organ or injury site. Depletion in vivo may be systemic or it may be confined to a particular area, such as an organ or wound.

Compositions for depletion of SAP may include those supplied directly to the sample. For instance all of the binding agents mentioned above may be supplied directly to the sample. They may be supplied in any form or formulation although those that do not substantially interfere with desired outcomes for the sample may be preferred.

Compositions for the depletion of SAP may also be produced in the sample, or in an organism containing the sample. For example, a transgene encoding an anti-SAP antibody may be introduced into the sample.

SAP may be directly depleted by a material that binds or sequesters SAP, such as agarose, CPHPC, 4,6-pyruvate acetyl of beta-D-galactopyranose, phosphoethanolamine agarose, anti-SAP antibodies, DNA analogs and carbohydrate analogs.

Depletion may also occur by degradation or inactivation of SAP such as through the use of SAP-specific proteases.

Other compositions may increase the rate of uptake of SAP and this decrease its availability.

Finally, SAP levels may also be depleted by interfering with its initial production or increasing its degradation. In a specific embodiment, SAP levels may be depleted in vivo by administering a composition that inhibits SAP production. Because SAP is primarily produced in the liver, in vivo suppression of SAP production should be easily attained, but will be systemic. Compositions that interfere with SAP production may act upon a signaling pathway that modulates SAP production.

### Suppression of SAP Activity

The disclosure may also include compositions and methods for the suppression of SAP activity. Suppression may be in a sample and may occur in vitro or in vivo. Compositions may also include compositions supplied directly to a sample and those produced in the sample. Many such compositions may be SAP-binding compositions described above. In particular, compositions for the suppression of SAP activity may include antibodies selected as described above to bind to specific regions of SAP not homologous to CRP. Antibodies may also target the region of SAP that binds to FcγR or may compete with SAP for binding to the these receptors. Small peptides may also be able to block SAP binding to the FcγR or compete with SAP for binding to these receptors.

Compositions that suppress SAP activity may act by a variety of mechanisms including but not limited to: decreasing the ability of SAP proteins to interact with monocytes, decreasing the ability of SAP proteins to interact with cofactors or decreasing the level of such cofactors, and interfering with SAP-induced signaling in monocytes, such as a pathway triggered by SAP binding to an FcγR. This pathway is described in detail in Daeron, Marc, “Fc Receptor Biology”, Annu. Rev. Immunology 15:203-34 (1997). In an exemplary embodiment a portion of the pathway that is not shared with other signaling cascades or only a limited number of non-critical signaling cascades may be selected for interference to minimize side-effects. For example, a composition may interfere with the Fc pathway by blocking syk kinase.

### Effects of IL-4 and IL-13

Yet another aspect of the disclosure relates to compositions and methods for promoting fibrocyte formation by providing IL-4, IL-13 or a combination of the two to monocytes. The monocytes may be located in vitro or in vivo. IL-4 and IL-13 may be provided by an extraneous source, or endogenous production may be increased. More specifically, IL-4 or IL-13 may be provided at concentrations of between approximately 0.1 and 10 ng/ml.

### Uses for Modulating Fibrocyte Formation

Depletion or suppression of SAP or supply of IL-4 or IL-13 in a sample may be used to increase fibrocyte differentiation from monocytes. This effect has many uses both in vitro and in vivo. For example, in vitro increased fibrocyte formation may be useful in tissue engineering. Production of fibrocytes in areas requiring vascularization may induce angiogenesis. In vitro, increased differentiation of monocytes to form fibrocytes may also be used for internal tissue engineering or for inducing angiogenesis in areas in need of new vasculature.

Additionally, increasing differentiation of monocytes into fibrocytes in vivo may promote wound healing or may be used for cosmetic surgery applications. Wound healing may benefit, inter alia, from the ability of fibrocytes to further differentiate into other cells such as myofibroblasts and from angiogenic effects of fibrocytes as well as the from their ability to function as APCs, thereby assisting in prevention or control of infection.

Because of the ability of fibrocytes to function as APCs, areas of chronic infection or areas that are infected but not readily reached by the immune system, such as cartilage, may also benefit from increased monocyte differentiation into fibrocytes.

Because pancreatic tumors and adenocarcinomas show lower levels of fibrocytes, increasing differentiation of monocytes into fibrocytes in these tissues may help slow the tumor progression or aid in remission.

### Specific Example Formulations

Some compositions of the present disclosure may be provided in a variety of formulations.

In a specific example, the disclosure may include methods and compositions for promoting wound healing by depleting or suppressing SAP in the region of a wound. These wound healing compositions may include CPHPC, anti-SAP antibodies, 4,6-pyruvate acetyl of β-D-galactopyranose, such as found on high EEO agarose, ethanolamines, such as those found on phosphoethanolamine agarose, Ca²⁺, and combinations thereof. Cytokines such as IL-13, IL-4, FGF and TGFβ may be added to these compositions.

In many patients only localized SAP depletion or inhibition or interference with a SAP-modulated pathway may be desirable. Many compositions within the scope of the present disclosure may be administered locally to such patients. For instance, administration of a composition may be topical, such as in an ointment, cream, solid, spray, vapor aerosol or wound dressing. Such topical formulations may include alcohol, water, disinfectants, other volatile substances, or any other pharmaceutically active agents, such as antibiotics and anti-infective agents, or pharmaceutically acceptable carriers. Local administration may also be by localized injection of a composition alone or in combination with another pharmaceutically active agent or pharmaceutically acceptable carrier.

Patients for whom localized administration of compositions that increase monocyte differentiation into fibrocytes may be advisable include but are not limited to: mild to moderate burn patients; patients who have suffered lacerations, including those inflicted during surgical procedures; patients suffering from diabetic complications, such as ulcers; patients with venous ulcers, pressure ulcers, or areas of low circulation internally; patients with abrasions, minor contusions or puncture wounds; patients with bullet or shrapnel wounds; patients with open fractures; patients in need of tissue growth for tissue engineering or cosmetic reasons; and immunosuppressed, hemophiliac, or other patients who are likely to benefit from the more rapid healing of most wounds.

For patients with severe or numerous wounds or other disorders, more general administration of a composition to promote fibrocyte formation through an IV or other systemic injection may be appropriate. Patients for whom systemic administration of a SAP depleting or inhibiting agent may be helpful include, but are not limited to: severe burn patients; later stage peripheral arterial occlusive disease patients; and patients with general wound healing disorders.

Some formulations may be appropriate for local or systemic administration. Additionally, the therapeutic agent may be supplied in a solid form, such as a powder, then reconstituted to produce the formulation ultimately administered to a patient.

In an exemplary embodiment for the treatment of wound healing, high EEO agarose or phosphoethanolamine agarose may be administered as a would dressing. In this embodiment, the agarose may be at a concentration of approximately 1% (w/v) and may also contain approximately 5 mM CaCl₂. The wound dressing may be applied for any period of time. Although it may be applied continuously until the wound has closed (approximately two days or more), it may also only be applied for a short initial period, such as 12 hours. This initial removal of SAP from the wound may be sufficient to induce increased differentiation of monocytes into fibrocytes and improve wound healing.

In another exemplary embodiment, CPHPC may be administered systemically to promote healing of widespread or recalcitrant wounds. CPHPC has been previously administered in a range of 1.5 to 15 mg/kg/day by osmotic pumps in mice in amyloidosis experiments. CPHPC has also been administered in 1 mg/ml water concentrations in drinking water for mice. A 20 g mouse drinks approximately 3 ml of water per day, resulting in an intake of approximately 0.15 CPHPC mg/kg/day. Such ranges are therefore likely safe in humans to reduce SAP levels, although different ranges may provide optimal benefit for wound healing.

In other embodiments, the compositions may be provided in or on prosthetic devices, particularly surgically implanted prosthetic devices.

In some embodiments, the compositions may be provided in a slow-release gel or dressing, such as a plastic substrate. Compositions may also be provided as hydrogels.

### Monocyte Differentiation Assays

Another aspect of the disclosure relates to assays to detect the ability of a sample to modulate fibrocyte differentiation from monocytes. In serum-free medium, normal monocytes form fibrocytes in two to three days. Normal serum, blood or other biological fluids suppress the formation of fibrocytes from normal monocytes over a specific dilution range. Thus the assay may be used to test whether a sample can modulate differentiation of monocytes into fibrocytes in serum-free medium. It may also be used to determine whether sample monocytes differentiate normally into fibrocytes in serum-free medium and if they respond normally to serum, SAP or other factors affecting this differentiation.

In a specific embodiment, the assay may be used to determine whether a patient's biological fluid has a decreased or increased ability to suppress monocyte differentiation into fibrocytes. If suppression by SAP is to be tested, any biological fluid in which SAP is normally or transiently present may be used, including whole blood, serum, plasma, synovial fluid, cerebral spinal fluid and bronchial fluid. An increased ability to suppress monocyte differentiation may be indicative of a wound healing disorder or other disorders, or the propensity to develop such a disorder. Although in many patients an increased ability of a biological fluid to suppress fibrocyte formation may be due to low levels of SAP, this is not necessarily the case. SAP may be present at normal levels, but exhibit decreased suppressive activity due to defects in the SAP itself or the absence or presence of a cofactor or other molecule. Methods of determining the more precise nature of the suppression problem, such as use of ELISAs, electrophoresis, and fractionation will be apparent to one skilled in the art.

The methodology described above may also be used to determine whether certain potential drugs that affect fibrocyte differentiation may or may not be appropriate for a patient.

In another specific embodiment, the assay may be used to determine if a patient's monocytes are able to differentiate into fibrocytes in serum-free medium and if they respond normally to a biological fluid, SAP or another composition. More particularly, if a patient with wound healing problems appears to have normal levels of SAP, it may be advisable to obtain a sample of the patient's monocytes to determine if they are able to differentiate in the absence of serum or SAP.

Finally, in another specific example, the assay may be used to test the effects of a drug or other composition on monocyte differentiation into fibrocytes. The assay may be used in this manner to identify potential drugs designed to modulate fibrocyte formation, or it may be used to screen for any potential adverse effects of drugs intended for other uses.

During wound healing, some circulating monocytes enter the wound, differentiate into fibroblast-like cells called fibrocytes, and appear to then further differentiate into myofibroblasts, cells which play a key role in collagen deposition, cytokine release and wound contraction. The differentiation of monocytes into fibrocytes is inhibited by the serum protein Serum Amyloid P (SAP). Depleting SAP at a wound site thus may speed wound healing. SAP binds to some types of agarose in the presence of Ca²⁺. Human SAP was found to bind an agarose with a K_{D} of 7×10⁻⁸ M and a Bmax of 2.1 μg SAP/mg wet weight agarose. Mixing this agarose 1:5 w/v with 30 μg/ml human SAP (the average SAP concentration in normal serum) in a buffer containing 2 mM Ca²⁺ reduced the free SAP concentration to ˜0.02 μg/ml, well below the concentration that inhibits fibrocyte differentiation. Compared to hydrogel and foam dressings, dressings containing this agarose and Ca²⁺ significantly increased the speed of wound healing in partial thickness wounds in pigs. This suggests that agarose/Ca²⁺ dressings may be beneficial for wound healing in humans.

Wound healing compositions, systems, and methods may promote, facilitate, accelerate, and/or otherwise favorably modify healing of a wound according to some embodiments. A wound may include, in some embodiments, a skin wound. For example, a wound may include a burn, a laceration, an abrasion, a puncture, a skin ulcer. In some embodiments, wound healing may be associated with formation of some, little, or no scar tissue.

During wound healing, some circulating monocytes present within the blood are attracted to the wound, where they differentiate into fibroblast-like cells called fibrocytes and at least in part mediate tissue repair. Fibrocytes express markers of both hematopoietic cells (CD45, MHC class II, CD34) and stromal cells (collagen I and III and fibronectin). Fibrocyte precursors appear to be a ˜10% subpopulation of CD 14+ peripheral blood monocytes.

Fibrocytes are largely absent from normal skin but are present in scars in both humans and mice, suggesting that fibrocytes participate in wound healing. Interestingly, the number of fibrocytes in hypertrophic scars is higher than in normal scars. Mature fibrocytes exposed to TGF-β in vitro are able to further develop into myofibroblasts, a population of fibroblast-like cells that are able to contract collagen gels, an in vitro model of wound contraction. Fibrocytes from burn patients secrete TGF-β to activate dermal fibroblasts, indicating that fibrocytes can have a multiplicative effect on wound healing.

A factor in serum was found that inhibits the differentiation. of monocytes into fibrocytes. The component of human serum that inhibits human fibrocyte differentiation was purified and identified as serum amyloid P (SAP). SAP is a 27 kDa protein produced by the liver, secreted into the blood, and circulates as stable 135 kDa pentamers. SAP binds to apoptotic cells, DNA and some micro-organisms and is cleared by macrophages and other cells through Fc gamma receptors. A commercial preparation of SAP has been identified that was able to inhibit fibrocyte differentiation, whereas the highly related protein C-reactive protein (CRP) could not. To confirm that SAP is the active factor in serum that inhibits fibrocyte differentiation, SAP was depleted from serum using anti-SAP antibodies bound to protein G beads. The SAP-depleted serum had a poor ability to inhibit fibrocyte differentiation. Together with the ability of purified SAP to inhibit fibrocyte differentiation, these observations strongly suggested that SAP is the active factor in serum that inhibits fibrocyte differentiation.

Agarose is a polysaccharide polymer isolated from seaweed. Different preparations of agarose contain different amounts of pyruvate acetal adducts and covalently linked sulfate. SAP binds strongly to some, but not all, types of agarose in the presence of millimolar levels of Ca²⁺. A comparison of the SAP binding capacity of different commercial agarose preparations in the presence of 2 mM CaCl₂ showed a correlation with pyruvate acetal content but no correlation with the agarose sulfate content.

Wound fluid contains serum proteins. Since SAP is present in serum at a concentration of ˜30 μg/ml and inhibits fibrocyte differentiation at ˜1 μg/ml or lower, there is a strong possibility that wound fluids initially contain enough SAP to inhibit fibrocyte differentiation. In support of this, we found that both systemic and local injections of murine SAP inhibit dermal wound healing in mice. We reasoned that a material that binds SAP might be able to deplete SAP at a wound site and thus potentiate fibrocyte differentiation and wound healing. In this report we show that a wound-healing dressing that contains a SAP-binding agarose and 2 mM CaCl₂ speeds wound healing in pigs.

The following examples are included to demonstrate specific embodiments of the disclosure. It should be appreciated by those of skill in the art that the techniques disclosed in the examples that follow represent techniques discovered by the inventors to function well in the practice of the disclosure. However, those of skill in the art should, in light of the present disclosure, appreciate that many changes can be made in the specific embodiments that are disclosed and still obtain a like or similar result without departing from the spirit and scope of the disclosure.

### EXAMPLES

Some specific example embodiments of the disclosure may be illustrated by one or more of the examples provided herein.

### Example 1

### Inhibition of Fibrocyte Formation

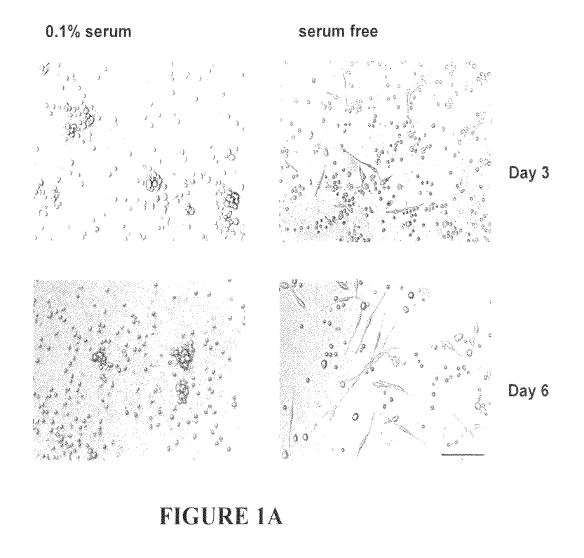

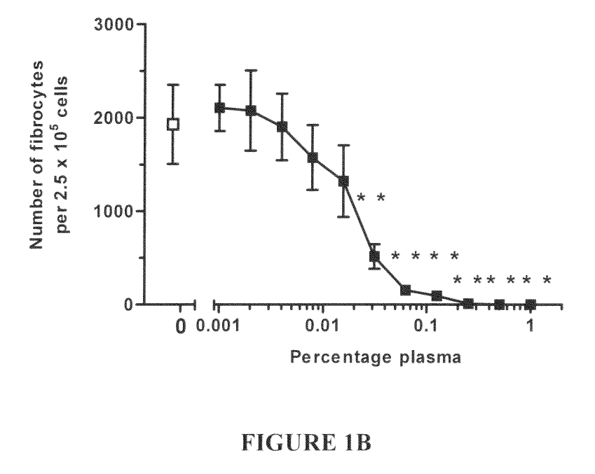

While examining the possible role of cell density in the survival of peripheral blood T cells, it was observed that in serum-free medium PBMC gave rise to a population of fibroblast-like cells. These cells were adherent and had a spindle-shaped morphology (FIG. 1A). Approximately 0.5-1% of PBMC differentiated into fibroblast-like cells in serum-free medium, and this occurred in tissue culture treated plasticware and borosilicate and standard glass slides.

The rapid appearance of these cells, within 3 days of culture, was inhibited by human serum or plasma. To examine this process in more detail, PBMC were cultured at 2.5×10⁵ cells per ml in serum-free medium containing increasing concentrations of human plasma for 6 days. When plasma was present at concentrations between 10% and 0.5%, the fibroblast-like cells did not differentiate (FIG. 1B). However, at or below 0.1% serum, fibroblast-like cells rapidly developed. The activity in the serum that inhibited fibrocyte formation was retained by a 30 kDa cutoff spin-filter (data not shown). If serum was heated to 56° C. for 30 minutes, the efficacy was reduced 10 fold, and heating to 95° C. abolished the inhibitory activity (data not shown).

These data suggested that that the inhibitory factor is a protein. As the inhibitory factor was present in human serum, it indicated that the activity was unlikely to be involved with the coagulation system. The inhibitory factor also appeared to be an evolutionary conserved protein as bovine, equine, caprine, and rat sera were also able to inhibit the appearance of these fibroblast-like cells (data not shown).

### Example 2

### Characterization of Fibroblast-Like Cells

The differentiation of these fibroblast-like cells from peripheral blood suggested that they might be peripheral blood fibrocytes. Fibrocytes are a population derived from peripheral blood monocytes that differentiate in vitro and in vivo into fibroblast-like cells. They rapidly enter wound sites and are capable of presenting antigens to T cells. Their phenotype is composed of both haematopoietic markers, such as CD45 and MHC class II, and stromal markers, such as collagen I and fibronectin. However in order to identify these cells, PBMC were generally cultured for 1-2 weeks in medium containing serum.

To characterize whether the cells observed in the system were fibrocytes, PBMC were depleted of T cells with anti-CD3, B cells with anti-CD19, monocytes with anti-CD14 or all antigen presenting cells with anti-HLA class II and then cultured in serum-free conditions for 6 days. Depletion of PBMC with anti-CD3 or anti-CD 19 did not deplete fibroblast-like cells from PBMC when cultured in serum-free cultures (data not shown). Depletion of antigen presenting cells with anti-HLA class II or monocytes with anti-CD14 antibody did prevent the appearance of fibroblast-like cells, indicating that the fibroblast-like cells are derived from monocytes and not a dendritic cell population.

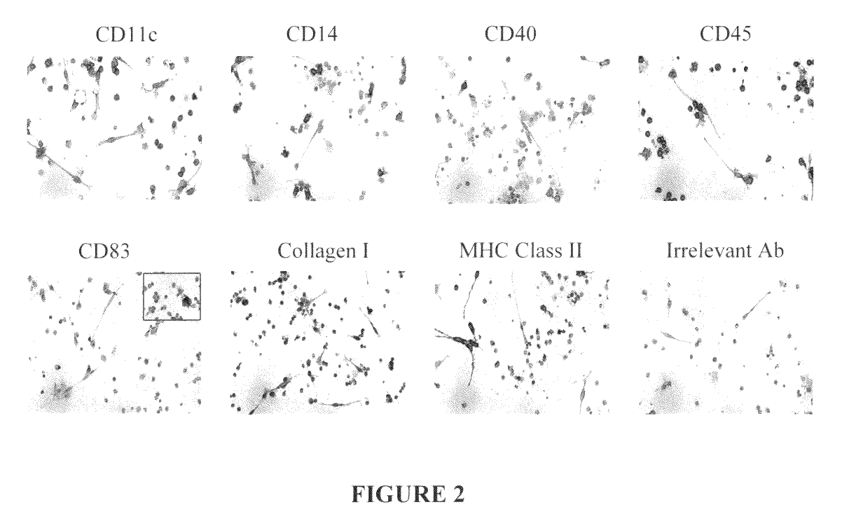

To further characterize the fibroblast-like cells, PBMC were cultured in serum-free medium for 5 days on glass slides. Cells were then air-dried, fixed in acetone and labeled with a variety of antibodies (Table 1 and FIG. 2). Fibrocytes express CD11a, CD11b, CD45, CD80, CD86, MHC class II, collagen I, fibronectin, the chemokine receptors CCR3, CCR5, CCR7, CXCR4 and α-smooth muscle actin. In the above culture conditions, the fibroblast-like cells in the present experiment also expressed all these markers. Fibrocytes are negative for CD1a, CD3, CD19, CD38 and vWF, as were the fibroblast-like cells in the present experiment. Based on these data it appears that the fibroblast-like cells observed in the experiments were fibrocytes. Further experiments were conducted to extend this phenotype. In the above conditions, the fibrocytes expressed several β1 integrins including α1 (CD49a), α2 (CD49b), α5 (CD49e), β1 (CD29) and β3 (CD61) along with high levels of β2 (CD 18), but were negative for α3, α4, α6 α4β7, αE and CLA (FIG. 2 and Table 1).

TABLE 1
Expression of surface markers on Fibrocytes

| | | Fibrocyte |
|---|---|---|
| Marker | Alternative Name | Expression |
| CD11a | LFA-1 | positive |
| CD11b | Mac-1 | positive |
| CD11c | | positive |
| CD13 | | positive |
| CD18 | β2 integrin | positive |
| CD29 | β1 integrin | positive |
| CD34 | | positive |
| CD40 | | weak positive |
| CD45 | LCA | positive |
| CD49a | α1 integrin | weak positive |
| CD49b | α2 integrin | negative |
| CD49e | α5 integrin | positive |
| CD51 | | positive |
| CD54 | ICAM-1 | positive |
| CD58 | LFA-3 | positive |
| CD61 | β3 integrin | positive |
| CD80 | B7-1 | weak positive |
| CD86 | B7-2 | positive |
| CD105 | Endoglin | positive |
| CD148 | | positive |
| MHC class II | | positive |
| CD162 | PSGL-1 | positive |
| CCR1 | | weak positive |
| CCR3 | | weak positive |
| CCR4 | | weak positive |
| CCR5 | | weak positive |
| CCR7 | | weak positive |
| CCR9 | | weak positive |
| CXCR1 | | positive |
| CXCR3 | | positive |
| CXCR4 | | weak positive |
| Collagen I | | positive |
| Collagen III | | positive |
| Fibronectin | | positive |
| α Smooth Muscle | | positive |
| Actin | | |
| Vimentin | | positive |
| CD1a | | negative |
| CD3 | | negative |
| CD10 | | negative |
| CD14 | | negative |
| CD19 | | negative |
| CD25 | | negative |
| CD27 | | negative |
| CD28 | | negative |
| CD38 | | negative |
| CD49c | α3 integrin | negative |
| CD49d | α4 integrin | negative |
| CD49f | α6 integrin | negative |
| CD69 | | negative |
| CD70 | CD27-L | negative |
| CD90 | | negative |
| CD103 | αE integrin | negative |
| CD109 | | negative |
| CD154 | CD40-L | negative |
| α4β7 | | negative |
| CLA | | negative |
| CCR2 | | negative |
| CCR6 | | negative |
| CXCR2 | | negative |
| CXCR5 | | negative |
| CXCR6 | | negative |
| Cytokeratin | | negative |
| vWF | | negative |

To obtain the data in Table 1, PBMC were cultured in the wells of 8 well glass slides at 2.5×10⁵ cells per ml (400 μl per well) in serum-free medium for 6 days. Cells were then air dried, fixed in acetone and stained by immunoperoxidase. Cells were scored positive or negative for the indicated antigens, compared to isotype-matched control antibodies.

### Example 3

### Characterization of the Fibrocyte Inhibitory Factor

The initial characterization of the serum factor that prevents rapid fibrocyte differentiation indicated that the factor was a heparin-binding molecule that eluted off an ion exchange column (High Q) as one of four proteins. By sequencing tryptic fragments of protein in a band cut from a native gel, one of these proteins was identified as C4b-binding protein (C4BP). C4b-binding protein is a 570 kDa protein, composed of seven alpha chains (70 kDa) and usually a single beta chain (40 kDa), which is involved in regulating the decay of C4b and C2a components of the complement system. C4BP also interacts with the vitamin K-dependent anticoagulant protein S. The C4BP/Protein S complex can be purified from serum or plasma using BaCl₂ precipitation.

To assess whether C4BP, or an associated protein, was the factor responsible for inhibiting fibrocyte differentiation, citrated plasma was treated with BaCl₂. The inhibitory factor was present in the BaCl₂ precipitate (FIG. 3 and Table 2). This fraction was applied to a heparin column and the fractions, eluted by increasing concentrations of NaCl, were assessed for their ability to inhibit monocyte to fibrocyte differentiation in serum free medium. The active factor was eluted off the heparin column in a peak at 200 mM NaCl (FIG. 3 and Table 2).

The fractions from the 200 mM peak were pooled and further fractionated by High Q ion exchange chromatography. A small peak eluting at 300 mM NaCl contained activity that inhibited fibrocyte differentiation. Analysis of the proteins present in this fraction indicated that the major band was a 27 kDa protein. Although the ion exchange chromatography led to a reduction in the amount of SAP recovered (FIG. 3A, lanes 8-10 and FIG. 3D, lanes 8-10) this step did remove several contaminating proteins. After the ion exchange step the only discernable contaminant was albumin at 65 kDa (FIG. 3A, lane 10).

The high Q fraction was concentrated and fractionated by electrophoresis on a non-denaturing polyacrylamide gel, followed by elution of the material in gel slices. A single band that migrated at approximately 140 kDa was able to inhibit differentiation (FIG. 3B). This band had a molecular weight of 27 kDa on a reducing polyacrylamide gel, suggesting that the native conformation of the protein was a pentamer (FIG. 3C). This band was excised from the gel, digested with trypsin and analyzed by MALDI mass spectrometry. Three major and two minor peptides were identified: VFVFPR, VGEYSLYIGR, AYSLFSYNTQGR, QGYFVEAQPK and IVLGQEQDSYGGK. These sequences exactly matched amino acid sequences 8-13, 68-77, 46-57, 121-130 and 131-143 of serum amyloid P.

To confirm that the active fractions contained SAP, fractions collected from column chromatography were analyzed by western blotting (FIG. 3D). The presence of SAP at 27 kDa was detected in all fractions that inhibited fibrocyte differentiation (FIG. 3D, lanes 6, 8, 10 and 11). A considerable amount of SAP was present in the supernatant from the BaCl₂ precipitation step indicating that this procedure was inefficient, with the recovery of only approximately 10-15% of the fibrocyte inhibitory activity in the BaCl₂ pellet (FIG. 3A lane 2). In order to remove the known problem of anti-SAP antibodies binding to immunoglobulins when used with western blotting, the antibody was pre-incubated with human IgG bound to agarose. Fractions were also analyzed for the presence of CRP, C4BP and protein S. Western blotting indicated that C4BP and Protein S were present in plasma, and in the barium precipitation, but were absent from the active fractions collected from heparin chromatography (data not shown).

TABLE 2
Recovery of protein and fibrocyte inhibitory activity
from fractionated human plasma

| | Volume | Protein | Total protein | |
|---|---|---|---|---|
| | (ml) | (mg/ml) | (mg) | Yield (%) |
| Plasma | 250 | 70 | 17,500 | 100 |
| BaCl₂ supernatant | 240 | 60 | 14,400 | 82.3 |
| BaCl₂ precipitate | 31 | 1 | 31 | 0.18 |
| Heparin fraction | 4.3 | 0.25 | 1.075 | 0.006 |
| High Q fraction | 1.96 | 0.05 | 0.098 | 0.00056 |
| Gel slice | 0.075 | 0.025 | 0.0018 | 0.00001 |

| | Activity | Total | Yield | Specific activity |
|---|---|---|---|---|
| | (U/ml) | activity (U) | (%) | (U/mg) |
| Plasma | 10,000 | 2.5 × 10⁶ | 100 | 143 |
| BaCl₂ supernatant | 6,666 | 1.6 × 10⁶ | 64 | 111 |
| BaCl₂ precipitate | 1,666 | 5.1 × 10⁴ | 2 | 1,645 |
| Heparin fraction | 500 | 2,150 | 0.086 | 2000 |
| High Q fraction | 400 | 720 | 0.029 | 7,300 |
| Gel slice | 2000 | 150 | 0.006 | 80,000 |

Plasma was fractionated by BaCl₂ precipitation, heparin and ion exchange chromatography. Protein concentrations were assessed by spectrophotometry at 280 nm. Inhibition of fibrocyte differentiation was assessed by morphology. The fibrocyte inhibitory activity of a sample was defined as the reciprocal of the dilution at which it inhibited fibrocyte differentiation by 50%, when added to serum-free medium.

SAP may also be detected by ELISA using the following methodology:

Maxisorb 96 well plates (Nalge Nunc International, Rochester, N.Y.) were coated overnight at 4° C. with monoclonal anti-SAP antibody (SAP-5, Sigma) in 50 mM sodium carbonate buffer pH 9.5. Plates were then incubated in Tris buffered saline pH 7.4 (TBS) containing 4% BSA (TBS-4% BSA) to inhibit non-specific binding. Serum and purified proteins were diluted to 1/1000 in TBS-4% BSA, to prevent SAP from aggregating and incubated for 60 minutes at 37° C. Plates were then washed in TBS containing 0.05% Tween-20. Polyclonal rabbit anti-SAP antibody (BioGenesis) diluted 1/5000 in TBS-4% BSA was used as the detecting antibody. After washing, 100 pg/ml biotinylated goat F(ab)₂ anti-rabbit (Southern Biotechnology Inc.) diluted in TBS-4% BSA was added for 60 minutes. Biotinylated antibodies were detected by ExtrAvidin peroxidase (Sigma). Undiluted peroxidase substrate 3,3,5,5-tetramethylbenzidine (TMB, Sigma) was incubated for 5 minutes at room temperature before the reaction was stopped by 1N HCl and read at 450 nm (BioTek Instruments, Winooska, Vt.). The assay was sensitive to 200 pg/ml.

### Example 4

### Specificity of Serum Amyloid P

Serum amyloid P is a constitutive plasma protein and is closely related to CRP, the major acute phase protein in humans. To assess whether other plasma proteins could also inhibit the differentiation of fibrocytes, PBMC were cultured in serum-free medium in the presence of commercially available purified SAP, CRP, C4b or Protein S. The commercially available SAP was purified using calcium-dependent affinity chromatography on unsubstituted agarose. Of the proteins tested, only SAP was able to inhibit fibrocyte differentiation, with maximal inhibitory activity at 1 μg/ml (FIG. 4). A dilution curve indicated that the commercially available SAP has approximately 6.6×10³ units/mg of activity (FIG. 4). Serum and plasma contain between 30-50 μg/ml SAP. Fibrocytes began to appear at a plasma dilution of 0.5%, which would be approximately 0.15-0.25 μg/ml SAP, which is comparable to the threshold concentration of purified SAP. The data showing that SAP purified using two different procedures inhibits fibrocyte differentiation strongly suggests that SAP inhibits fibrocyte differentiation.

Although these data indicate that SAP is capable of inhibiting fibrocyte development and SAP purifies in a manner that indicates that it is the active factor in plasma, it was not determined whether depletion of SAP from plasma and serum would negate the inhibition. Accordingly, SAP was depleted from plasma using agarose beads (BioGel A, BioRad). Plasma was diluted to 20% in 100 mM Tris pH 8, 150 mM NaCl, 5 mM CaCl₂ buffer and mixed with 1 ml agarose beads for 2 hours at 4° C. Beads were then removed by centrifugation and the process repeated. This depleted plasma was then assessed for its ability to inhibit fibrocyte differentiation. The control plasma diluted to 20% in 100 mM Tris pH 8, 150 mM NaCl, 5 mM CaCl₂ buffer had a similar dilution curve to that observed with untreated plasma. In contrast, the bead-treated plasma was less able to inhibit fibrocyte differentiation at intermediate levels of plasma. These data, along with the ability of purified SAP to inhibit fibrocyte differentiation, strongly suggest that SAP is the active factor in serum and plasma that inhibits fibrocyte differentiation. (See FIG. 5A).

Plasma was also depleted of SAP using protein G beads coated with anti-SAP antibodies. Removal of SAP led to a significant reduction in the ability of plasma to inhibit fibrocyte differentiation compared with plasma, or plasma treated with beads coated with control antibodies (p<0.05) (FIG. 5B). The beads coated with control antibodies did remove some of the fibrocyte-inhibitory activity from plasma, but this was not significantly different from cells cultured with plasma. This probably reflects SAP binding to the agarose in the protein G beads. These data, together with the ability of purified SAP to inhibit fibrocyte differentiation, strongly suggest that SAP is the active factor in serum and plasma that inhibits fibrocyte differentiation.

### Example 5

### Antibodies and Proteins

Purified human CRP, serum amyloid P, protein S and C4b were purchased from Calbiochem (San Diego, Calif.). Monoclonal antibodies to CD1a, CD3, CD11a, CD11b, CD11c, CD14, CD16, CD19, CD34, CD40, Pan CD45, CD64, CD83, CD90, HLA-DR/DP/DQ, mouse IgM, mouse IgG1 and mouse IgG2a were from BD Pharmingen (BD Biosciences, San Diego, Calif.). Chemokine receptor antibodies were purchased from R and D Systems (Minneapolis, Minn.). Rabbit anti-collagen I was from Chemicon International (Temecula, Calif.), monoclonal C4b-binding protein was from Green Mountain Antibodies (Burlington, VE), sheep anti human C4b-binding protein was from The Binding Site (Birmingham, UK), monoclonal anti-CRP was from Sigma (St. Louis, Mo.). Polyclonal rabbit anti-protein S was from Biogenesis (Poole, Dorset, UK).

### Example b 6:

### Cell Separation

Peripheral blood mononuclear cells were isolated from buffy coats (Gulf Coast Regional Blood Center, Houston, Tex.) by Ficoll-Paque (Amersham Biosciences, Piscataway, N.J., USA) centrifugation for 40 minutes at 400×g. Depletion of specified leukocyte subsets was performed using negative selection using magnetic Dynabeads (Dynal Biotech Inc., Lake Success, N.Y.), as described previously. Briefly, PBMC were incubated with primary antibodies for 30 minutes at 4° C. Cells were then washed and incubated with Dynabeads coated with goat anti-mouse IgG for 30 minutes, before removal of antibody-coated cells by magnetic selection. This process was repeated twice. The negatively selected cells were routinely in excess of 98% pure as determined by monoclonal antibody labeling.

### Example 7

### cell Culture and Fibrocyte Differentiation Assay

Cells were incubated in serum-free medium: RPMI (GibcoBRL Life, Invitrogen, Carlsbad, Calif., USA) supplemented with 10 mM HEPES (GibcoBRL/Life), 2 mM glutamine, 100 U/ml penicillin and 100 μg/ml streptomycin, 0.2% bovine serum albumin (BSA, Sigma), 5 μg/ml insulin (Sigma), 5 μg/ml iron-saturated transferrin (Sigma) and 5 ng/ml sodium selenite (Sigma). Normal human serum (Sigma), normal human plasma (Gulf Coast Regional Blood Center) or fetal calf serum (Sigma), column fractions, sera and synovial fluid from patients or purified proteins were added at the stated concentrations.

PBMC were cultured in 24 or 96 well tissue culture plates in 2 ml or 200 μl volumes respectively (Becton Dickinson, Franklin Lakes, N.J.) at 2.5×10⁵ cells per ml in a humidified incubator containing 5% CO₂ at 37° C. for the indicated times. Fibrocytes in 5 different 900 μm diameter fields of view were enumerated by morphology in viable cultures as adherent cells with an elongated spindle-shaped morphology as distinct from small lymphocytes or adherent monocytes. Alternatively cells were air dried, fixed in methanol and stained with haematoxylin and eosin (Hema 3 Stain, VWR, Houston, Tex.). Fibrocytes were counted using the above criterion and the presence of an oval nucleus. Enumeration of fibrocytes was performed on cells cultured for 6 days in flat-bottomed 96 well plates, with 2.5×10⁴ cells per well. In addition, fibrocyte identity was confirmed by immunoperoxidase staining (see below). The fibrocyte inhibitory activity of a sample was defined as the reciprocal of the dilution at which it inhibited fibrocyte differentiation by 50%, when added to serum-free medium.

### Example 8

### Purification and Characterization of Serum and Plasma Proteins

100 ml of frozen human serum or plasma was thawed rapidly at 37° C. and 1× “Complete” protease inhibitor (Roche, Indianapolis, Ind., USA), 1mM benzamidine HCl (Sigma) and 1mM Pefabloc (AEBSF: 4-(2-Aminoethyl)-benzenesulfonyl fluoride hydrochloride, Roche) were added. All subsequent steps were performed on ice or at 4° C. Barium citrate adsorption of plasma was performed as described previously. The precipitate was collected by centrifugation at 10,000×g for 15 minutes, resuspended in 20 ml of 100 mM BaCl₂ plus inhibitors and recentrifuged. After two rounds of washing, the pellet was resuspended to 20 ml in 10 mM sodium phosphate buffer pH 7.4 containing 5 mM EDTA and 1 mM benzamidine HCl and dialyzed for 24 hours against three changes of 4 liters of the same buffer.

Chromatography was performed using an Econo system (Bio-Rad, Hercules, Calif.) collecting 1 ml samples with a flow rate of 1 ml/min. The dialyzed barium citrate precipitate was loaded onto a 5 ml Hi-Trap Heparin column (Amersham Biosciences) and the column was washed extensively in 10 mM sodium phosphate buffer pH 7.4 until the absorbance at 280 nm returned to baseline. Bound material was eluted with a stepped gradient of 15 mls each of 100, 200, 300 and 500 mM NaCl in 10 mM sodium phosphate buffer pH 7.4. The fractions that inhibited monocyte to fibrocyte differentiation eluted at 200 mM NaCl. These were pooled (2 ml) and loaded onto a 5 ml Econo-Pak High Q column. After washing the column in 10 mM phosphate buffer, the bound material was eluted with the stepped gradient as above, with the active fraction eluting at 300 mM NaCl.

Active fractions from the High Q chromatography were concentrated to 200 μl using Aquacide II (Calbiochem) and then loaded onto a 4-20% native polyacrylamide gels (BMA, BioWhittaker, Rockland, Me.) as described previously. After electrophoresis, gel lanes were cut into 5 mm slices, mixed with 200 μl 20 mM sodium phosphate, 150 mM NaCl, 5 mM EDTA pH 7.4 containing 1 mM benzamidine HCl, crushed with a small pestle in an eppendorf tube and placed on an end-over-end mixer at 4° C. for 3 days. Proteins that eluted from the gel were analyzed for activity. To obtain amino acid sequences, proteins eluted from the gel slices were loaded onto a 4-20% gel with 100 μM thioglycolic acid (Sigma) in the upper chamber. After electrophoresis the gel was rapidly stained with Coomasie brilliant blue, destained, and the bands excised off the gel. Amino acid sequencing was performed by Dr Richard Cook, Protein Sequencing Facility, Department of Immunology, Baylor College of Medicine.

### Example 9

### Western Blotting

For western blotting, plasma and serum samples were diluted 1:500 in 10 mM sodium phosphate pH 7.4. Fractions from heparin and High Q columns were not diluted. Samples were mixed with Laemmeli's sample buffer containing 20 mM DTT and heated to 100° C. for 5 minutes. Samples were loaded onto 4-20% Tris/glycine polyacrylamide gels (Cambrex). Samples for native gels were analyzed in the absence of DTT or SDS. Proteins were transferred to PVDF (Immobilon P, Millipore, Bedford, Mass.) membranes in Tris/glycine/SDS buffer containing 20% methanol. Filters were blocked with Tris buffered saline (TBS) pH 7.4 containing 5% BSA, 5% non-fat milk protein and 0.1% Tween 20 at 4° C. for 18 hours. Primary and biotinylated secondary antibodies were diluted in TBS pH 7.4 containing 5% BSA, 5% non-fat milk protein and 0.1% Tween 20 using pre-determined optimal dilutions (data not shown) for 60 minutes. ExtrAvidin-peroxidase (Sigma) diluted in TBS pH 7.4 containing 5% BSA and 0.1% Tween 20 was used to identify biotinylated antibody and chemiluminescence (ECL, Amersham Biosciences) was used to visualize the result.

### Example 10

### Immunohistochemistry

Cells cultured on 8 well glass microscope slides (Lab-Tek, Nalge Nunc International, Naperville, Ill.) were air dried before fixation in acetone for 15 minutes. Endogenous peroxidase was quenched for 15 minutes with 0.03% H₂O₂ and then non-specific binding was blocked by incubation in 2% BSA in PBS for 60 minutes. Slides were incubated with primary antibodies in PBS containing 2% BSA for 60 minutes. Isotype-matched irrelevant antibodies were used as controls. Slides were then washed in three changes of PBS over 15 minutes and incubated for 60 minutes with biotinylated goat anti-mouse Ig (BD Pharmingen). After washing, the biotinylated antibodies were detected by ExtrAvidin peroxidase (Sigma). Staining was developed with DAB (Diaminobenzadine, Sigma) for 3 minutes and counterstained for 30 seconds with Mayer's haemalum (Sigma).

### Example 11

### Expression of Surface Makers on Fibrocytes

PBMC were cultured in the wells of 8 well glass slides at 2.5×10⁵ cells per ml (400 μl per well) in serum-free medium for 6 days. Cells were then air dried, fixed in acetone and stained by immunoperoxidase. Cells were scored positive or negative for the indicated antigens, compared to isotype-matched control antibodies.

### Example 12

### Recovery of Protein and Fibrocyte Inhibitory Activity From Factionated Human Plasma

Plasma was fractionated by BaCl₂ precipitation, heparin and ion exchange chromatography. Protein concentrations were assessed by spectrophotometry at 280 nm. Inhibition of fibrocyte differentiation was assessed by morphology. The fibrocyte inhibitory activity of a sample was defined as the reciprocal of the dilution at which it inhibited fibrocyte differentiation by 50%, when added to serum-free medium.

### Example 13

### Rat Wound Healing Studies Using High EEO Agarose Bandages

One application of the present disclosure relates to treatment of small wounds such as small cuts and surgical incisions as well as chronic ulcers, such as diabetic ulcers. Treatments developed for these and similar applications may also be readily modified for treatment of larger wounds and more serious problems.

Local depletion of SAP is important in wound healing and experiments such as those described above have revealed that SAP binds particularly well to a type of agarose known in the art as high EEO agarose. This binding has also been determined to be influenced by the presence of calcium. To test the effects of a calcium/agarose bandage on wound healing, 4 cm wounds through the entire thickness of skin were made on the backs of three anesthetized rats. (See FIG. 6.) There was little bleeding from the wounds. One rat was treated only with a 4×4 gauze bandage (Topper 4×4 sponge gauze, Johnson & Johnson, Skillman, N.J.) lightly soaked with 1 ml saline solution (0.9% NaCl w/v in water). This layer of gauze was covered with a dry 4×4 gauze bandage, and these were held in place with several layers of Vetwrap® (3M Animal Care Products, St. Paul, Minn.) which were wrapped around the torso of the rat. A second rat was treated with a similar bandage, with the first layer lightly soaked (1 ml) with saline/5 mM CaCl₂.

A third rat was treated with an agarose/CaCl₂ bandage. To make the first layer of this bandage, 0.2 g of high EEO agarose (Electrophoresis grade high EEO Agarose product # BP-162, Fisher Scientific, Fair Lawn, N.J.) was dissolved in 20 ml of the saline/CaCl₂ solution described above by heating the solution in a 50 ml polypropylene tube (Falcon, Becton Dickinson, Franklin Lakes, N.J.) in a microwave oven until the mixture began to boil. After swirling to dissolve the agarose, 1 ml of the hot mixture was poured on a 4×4 gauze bandage that was laying flat on a piece of plastic wrap. The agarose-CaCl₂-saline impregnated gauze bandage was allowed to cool. This was then used as the first layer of the bandage for the third rat. A second layer of dry gauze and a cover of Vetwrap® were applied as in the first two rats.

Each rat was separately anesthetized, photographed, and bandaged to minimize differences in time between anesthetizing, wounding and bandaging.

After 24 hours, the rats were lightly anesthetized and weighed, then the bandages were removed and the wounds were photographed. (See FIG. 7.) New bandages of the same initial composition were then reapplied to each of the rats. After another 24 hours this process was repeated to obtain additional pictures. (See FIG. 8.)

The rat treated with the agarose/CaCl₂ bandage showed considerably more rapid wound healing than either of the other two rats. (See FIG. 8B.)

Although an agarose bandage was reapplied each day in the present example, in other embodiments of the disclosure an agarose bandage may be applied only initially or initially and on the first day or so followed by a dry bandage once the wound has substantially closed. Once the wound has closed, the ability of agarose to absorb SAP may be limited. Wounds that have closed may also benefit from a dryer environment.

Although hydrated agarose was used in the present example, it may be possible to also utilize bandages and other formulations with less hydrated agarose. The agarose may be wetted by serum escaping from the wound itself.

Topical agarose preparations of the present disclosure may also be prepared using antiseptics to allow both cleansing of the wound and promotion of wound healing. In a specific example, the agarose may be prepared with alcohol, which may cleanse the wound initially then evaporate over time.

### Example 14

### Additional Factors for Use in Topical Wound Healing Embodiments

Although the above agarose bandages proved quite effective in promoting wound healing, the observed effects can most likely be improved by the addition of other wound healing factors to the bandages or other topical agarose formulations. Such factors may include any compound or compositions, such as small molecules or polypeptides.

In particular, these factors may influence a separate wound healing pathway, or they may influence the fibrocyte formation pathway. They may also influence the fibrocyte formation pathway in a different manner than SAP, or they may influence it by a mechanism similar to that of SAP, for example antibodies in the agarose formulation may bind and inactivate additional SAP.

Factors may also be included that address other problems, some of which may also affect wound healing. For example, agarose bandages for hemophiliac patients may additionally include clotting factors to help stop or prevent bleeding from the wound.

In a particular embodiment, IL-4 and/or IL-13 may be included in the agarose formulation. Both are potent activators of the fibrotic response. IL-4 has been previously described to play a role in wound repair and healing.

Experiments have shown that IL-4 and IL-13 are capable of promoting fibrocyte differentiation in vitro. Specifically, PBMC were cultured in serum-free medium in the presence of IL-4 or IL-13. Concentrations of either IL-4 or IL-13 between 10 and 0.1 ng/ml enhanced the number of fibrocytes in culture. (See FIG. 9.) This indicates that IL-4 and IL-13 are capable of promoting the differentiation of fibrocyte precursors into mature fibrocytes. Therefore a bandage or other topical agarose formulation as described above additionally containing IL-4 and/or IL-13 is expected to show further improvements in wound healing.

Other factors that may be added to agarose bandages or topical formulations as described above or physiological conditions to be mimicked include:
Molecules known to bind to SAP:
Collagen IV;
Laminin;
Fibronectin;
C4BP;
Aggregated Fc of IgG;
CD16, CD32 and CD64: Fc Receptors;
Heparin;
LPS;
Apoptotic cells, especially chromatin and DNA
Zymozan.

Physiological conditions related to SAP binding:
SAP exhibits calcium-dependent binding to amyloid fibers formed from, e.g. serum amyloid A (SAA), immunoglobulin light chains, β2 microglobulin, transthyretin and the neurofibrillary tangles;
SAP binds to surfaces of bacteria due to expression of pyruvate acetyl of galactose and to other sugars on the surface of bacteria.
SAP binds to the “artificial” ligands on high EEO agarose and phosphoethanolamine-agarose, and with low affinity to phosphocholine-sepharose. These features give rise to two ways of purifying SAP from serum or plasma. First, SAP may be bound to high EEO agarose via the pyruvate acetyl of galactose, which is a minor constituent of agarose preparations. Second, SAP may be bound to phosphoethanolamine-agarose, which is presently the preferred method of SAP purification in the art. Thus, phosphoethanolamine-agarose may be used for bandages or topical formulations.

### Example 15

### Methods of Identifying Suitable Sap-Binding Agents Including Derivatized Agarose

Because the biological function of SAP includes opsonization of foreign molecules for enhanced uptake by macrophages, other derivatized agaroses incorporating motifs such as bacterial cell wall carbohydrates, DNA or DNA analogs, and the like may also be used if they meet the following criterion for activity.

Prepare a 100 microliter sample of SAP at 20 micrograms/milliliter. Add insoluble adsorbent in an amount that increases the volume of the sample by less than 100%. Incubate with gentle shaking or end over end rotation for 1 hour. Centrifuge to pellet the adsorbent. Measure remaining SAP in the supernatant. If more than 50% of the SAP has been removed, the adsorbent is deemed active.

The methodology may also be used to identify and test other SAP-binding agents.

The methodology was used to determine the calcium ion (Ca²⁺) concentrations at which one agarose sample was operable to bind SAP. The specific experimental protocol was as follows:

Pre-hydrated agarose beads (SP Sepharose FF, GE-Healthcare Biosciences, Uppsala, Sweden) were washed four times in 10 volumes of 10 mM Tris pH 8.0 (pH adjusted with HCl)/140 mM NaCl, collecting the beads by centrifugation at 2,000×g for 1 minute. For each assay point, 20 mg of beads were placed in a 1.5 ml Eppendorf tube. The beads were then washed three times in 1 ml of 10 mM Tris pH 8.0/140 mM NaCl containing different concentrations of calcium chloride (10, 5, 2, 1, 0.3, 0.1, 0.03 mM CaCl₂ and no calcium control). 100 μl of purified human SAP (30 μg/ml; EMD Biosciences, La Jolla, Calif.) in 10 mM Tris pH 8.0/140 mM NaCl containing different concentrations of calcium chloride (10, 5, 2, 1, 0.3, 0.1, 0.03 mM CaCl₂ and no calcium control) was then added to the agarose. The tube was rotated end over end for 60 minutes at room temperature, and the agarose beads were collected by centrifugation at 2,000×g for 1 minute. Supernatants were collected, and free SAP concentrations were determined by ELISA following Pilling et al. (2003). Inhibition of fibrocyte differentiation by serum amyloid P. Journal of Immunology 17: 5537-5546. The experiment was repeated three times and the cumulative results are shown in FIG. 17. This data shows that the agarose tested was able to efficiently depleted SAP from the solution, and thus bind SAP at a Ca²⁺ concentration of 0.3 mM and higher.

### Example 16

### Pig Wound Healing Studies Using Agarose Hydrogels

The effects of agarose hydrogels on deep partial thickness wound healing in a porcine model were also studied. The porcine model has morphological similarities to human skin. A total of seven young female specific pathogen free (SPF: Ken-O-Kaw Farms, Windsor, Ill.) pigs weighing 25-30 kg were maintained in constant conditions for two weeks prior to the experiment. These animals were fed a basal diet ad libitum and were housed individually in animal facilities in compliance with the American Association for Accreditation of Laboratory Animal Care with controlled temperature (19-21° C.) and lighting (12 hours light/12 hours dark).

The flank and back of the experimental animals were clipped with standard animal clippers on the day of the experiment. The skin on both sides of each animal was prepared for wounding by washing with a non-antibiotic soap (Neutrogena Soap Bar; Johnson & Johnson, Calif.) and sterile water. Each animal was anesthetized intramuscularly with tiletamine HCl plus zolazepam (1.4 mg/kg) (Telazol; Laderle Patenterals Inc., Puerto Rico), xylazine (2.0 mg/kg) (X-jet; Phoenix Scientific Inc., Mo.), and atropine (0.04 mg/kg) (Atrojet SA, Phoenix Scientific Inc., Mo.) followed by mask inhalation of isoflurane (Isothesia, Abbott Laboratories, Ill.) and oxygen combination.

One hundred and sixty (160) rectangular wounds measuring 10 mm×7 mm×0.5 mm were made in the paravertebral and thoracic area with a specialized electrokeratome fitted with a 7 mm blade. The wounds were separated from one another by 15 mm of unwounded skin.

Forty wounds were randomly assigned to a treatment group according to one of the three experimental designs. One animal in a preliminary study was assigned to a treatment group where wounds received either i)ME Agarose gel, ii) SP Agarose gel, ii) the vehicle alone, or iv) were untreated and exposed to air. Both ME Agarose and SP Agarose gels met the criteria stated in Example 15.

One other animal in the preliminary study was assigned to a treatment group where wounds received either i)SP Agarose gel, ii) the vehicle alone, iii) Vigilon wound dressing (C. R. Bard, Inc., Ga.) or iv) were untreated and exposed to air.

For the preliminary study, three animals were included. In these experiments two hydrogel test agents (SP and ME Agarose) along with positive and negative controls were evaluated. These treatments were randomized among these three animals with two of the animals receiving SP Agarose hydrogel material. Because it appeared that the SP material was more effective than the ME Agarose hydrogel, four additional animals were studied using the SP Agarose hydrogel alone.

Four animals were assigned to a treatment group where wounds received either i)SP Agarose gel, ii) the vehicle alone, iii) Vigilon, or iv) were untreated and exposed to air. All wounds in all treatment groups were covered with a polyurethane dressing except those that were untreated an exposed to air.

The application and assessment of different treatment groups is shown in FIG. 10. Areas A, B, C, and D are repeated areas of treatment.

All hydrogel treated wounds were treated by placing the hydrogel material over the wounds and surrounding normal skin to the approximate thickness of the Vigilon (˜1 mm). The hydrogel was then covered with a polyurethane dressing to prevent desiccation. One day 1 after treatment, the animals were anesthetized and the dressings observed to make sure they were still intact. All materials were kept in place until wound evaluation unless it was observed that the materials needed to be replaced. In order to assess the wounds, a portion of the hydrogel was removed to uncover five wounds for evaluation. Wounds were evaluated for epithelization as described below.

Animals were monitored daily for any observable signs of pain or discomfort. In order to help minimize possible discomfort, an analgesic buprenorphine 0.03 mg/kg (Buprenex injectable, Reckitt Benckiser Hull, England) was given to each animal on the first day, and every third day thereafter, while under anesthesia. A fentanyl transdermal system: 25 μg/hr (Duragesic; Alza Corp., Calif.) was used during the entire experiment.

Wounds were examined regularly for any signs of erythema (redness) and infection. The physical characteristics of the material were also noted.

Beginning on day 3 after wounding for the first 3 pigs in the preliminary study and on day 4 after wounding for the final 4 pigs and on each day thereafter until the time of healing (day 6 for most treatment groups), five wounds and the surrounding normal skin from each treatment group were excided using an electrokeratome with a 22 mm blade set to a depth of 0.7 mm. (See FIG. 10.) All specimens that were not excised intact were discarded. The excised skin containing the wound site was incubated in 0.5 M sodium bromide at 37° C. for 24 hours, allowing for a separation of the dermis from the epidermis. After separation, the epidermal sheet was examined macroscopically for defects. (FIG. 11.) Defects were defined as holes in the epidermal sheet or as a lack of epidermal continuity in the area of the wound. Epithelization was considered complete (healed) if no defect(s) were present; any defect in the wound area indicates that healing is incomplete.

The hydrogel materials did not cause any re-injury of wounds during removal throughout the entire assessment time. During the later evaluations (days 8-10) the hydrogel materials appeared to decrease in thickness (density) and became moderately desiccated, forming a glue-like substance.

The untreated air exposed wounds displayed prominent crust formation as compared to wounds from the other treatment groups. None of the wounds from any of the treatment groups showed signs of erythema or infection. Wounds treated with all hydrogel materials appeared to have significantly less crust formation as compared to wounds in the untreated air exposed group.

After the study was completed the number of wounds completely healed (completely epithelized) was divided by the total number of wounds sampled per day and multiplied by 100 to determine the % epilthelization (FIG. 12). The Chi square test was used to determine statistical significance between the treatment groups. Specific results by day were also examined. On day 3 none of the wounds in any treatment group were healed.

On day 4, 33% of the wounds treated with the SP Agarose hydrogel were healed. 13% of the wounds treated with the vehicle were healed. None of the other wounds were healed on that day.

On day 5, 90% of the wounds treated with SP Agarose gel were completely re-epithelized and 80% of the wounds in the vehicle group were healed. Wounds in the Vigilon group were 56% healed and 3% of the wounds in the untreated group were healed.

On day 6, 100% of the wounds treated with SP Agarose gel, Vigilon and the vehicle were healed. Only 40% of the untreated wounds were healed.

On day 7, 100% of the wounds from all treatment groups were completely re-epithelized except those from the untreated group, which were 60% healed.

On day 8, all wounds from each treatment group were completely re-epithelized.

These results collectively show that all hydrogel treatment groups increased the rate of epithelization as compared to untreated, air-exposed control wounds. These treatment groups initiated 100% complete epithelization two days earlier than the untreated, air-exposed wounds.

Further, wounds treated with the SP Agarose hydrogel healed significantly faster than wounds treated with Vigilon.

### Example 17

### SAP Binding

Pre-hydrated agarose beads (SP Sepharose FF, GE-Healthcare Biosciences, Uppsala, Sweden) were washed four times in 10 volumes of 10 mM Tris pH 8, 140 mM NaCl, 2 mM CaCl₂ (TNC buffer), collecting the beads by centrifugation at 2,000×g for 1 minute. For each assay point, 20 mg of beads were placed in a 1.5 ml Eppendorf tube. 100 μl of different concentrations of purified human SAP (EMD Biosciences, La Jolla, Calif.) in TNC was then added to the agarose. The tube was rotated end over end for 60 minutes at room temperature, and the agarose beads were collected by centrifugation at 2,000×g for 1 minute. Supernatants were collected, and SAP concentrations were determined by ELISA following Pilling et al. Because the free (supernatant) concentrations were equivalent to or less than the bound concentrations, bound amounts were calculated as total-free. Nonlinear regression to fit binding data to a standard one-site binding model was done with the Prism software package (GraphPad Software, San Diego, Calif.).

### Example 18

### Binding Specificity Assay

SP agarose was washed in TNC 4 times as described in Example 17 above. To examine binding specificity, 200 μl of human serum (Sigma, St. Louis, Mo.) or pig serum (a gift from Dr. Oluyinka Olutoye, Baylor College of Medicine) was mixed with 200 μl of SP agarose slurry and 600 μl of TNC. The mixture was rotated overnight at 4° C. The agarose beads were collected by centrifugation, washed 5 times in TNC, and bound material was eluted for 2 hours at room temperature with 200 μl of 10 mM Tris pH 8, 140 mM NaCl, 10 mM EDTA. The supernatant was clarified by centrifugation and 10 μ of the eluate was mixed with SDS sample buffer and separated on a 4-20% Tris/glycine gel (Biorad, Hercules, Calif.). The gel was then stained with Coomassie. To detect SAP, western blots were done following, using a 1:20,000 dilution of 1793-1 rabbit anti-SAP (Epitomics, Burlingame, Calif.) in TBS for the first antibody step.

High electroendoosmosis (high EEO) agarose binds SAP in the presence of Ca²⁺. After testing 11 different agarose sources, we identified SP Sepharose FF as having the highest specific binding to human SAP from serum (data not shown). A binding curve fit with a classic one-site binding model indicated that SP Sepharose FF binds human SAP with a KD of ˜9 μg/ml (7×10-8 M), and a Bmax of 42 μg SAP/20 mg wet weight agarose, or 2.1 μg SAP/mg wet weight agarose (FIG. 13A). Fits to a model with cooperative binding gave a Hill coefficient of 0.95, indicating no cooperative binding, and an F-test comparison to a two-site binding model indicated that there was no significant evidence for two-site binding. To estimate the possible effect of adding agarose to wound fluid containing different amounts of SAP, we measured the SAP concentration in a buffer containing SAP before and after adding a 1:5 w/v ratio of agarose beads to the solution. As shown in FIG. 13B, adding agarose decreases the free SAP concentration. At an initial human SAP concentration of 30 μg/ml (approximately the average concentration in human serum), the addition of 1:5 w/v agarose lowered the free SAP concentration to ˜0.02 μg/ml, well below the concentration that inhibits fibrocyte differentiation.

Porcine wounds are an excellent model for human wounds, and a topical application of high EEO agarose to deplete SAP from a porcine wound was tested. High EEO agarose binds human SAP with high specificity, and in fact can be used to purify SAP from serum. To determine if high EEO agarose will similarly bind porcine SAP, porcine serum was mixed with SP agarose, washed, and the bound material was eluted. As shown in FIG. 14A, high EEO agarose showed high affinity binding of a single 27 kDa protein in porcine serum, and this protein had essentially the same molecular mass as human SAP. A western blot stained with anti-human SAP antibodies suggested that the 27 kDa protein from porcine serum that bound to the SP agarose is SAP (FIG. 14B). These results suggest that high EEO agarose can be used to specifically absorb porcine SAP from porcine serum.

### Example 19

### Experimental Animals

Sixteen young female specific pathogen free (SPF: Ken-O-Kaw Farms, Windsor, Ill.) pigs weighing 25-30 kg were kept in the University of Miami animal facility (meeting American Association for Accreditation of Laboratory Animal Care (AAALAC) compliance) for two weeks prior to initiating the experiment. These animals were fed a basal diet ad libitum and were housed individually with controlled temperature (19-21° C.) and lighting (12 h/12 h LD). The experimental animal protocols used for this study were approved by the University of Miami Institutional Animal Care and Use Committee and all the procedures followed the federal guidelines for the care and use of laboratory animals (U.S. Department of Health and Human Services, U.S. Department of Agriculture). The studies were conducted in compliance with the University of Miami's Department of Dermatology and Cutaneous Surgery Standard Operating Procedures. Animals were monitored daily for any observable signs of pain or discomfort. In order to help minimize possible discomfort, 0.03 mg/kg buprenorphine (Buprenex injectable; Reckitt Benckiser Hull, England) was given to each animal on the first day, and every third day thereafter, and a 25 μg/hr fentanyl transdermal system (Duragesic; Alza Corp. Mountain View, Calif.) was used during the entire experiment.

### Example 20

### Wounding Technique

The flanks and backs of experimental animals were clipped with standard animal clippers on the day of the experiment. The skin on both sides of each animal was prepared for wounding by washing with a non-antibiotic soap (Neutrogena Soap Bar; Johnson and Johnson, Los Angeles, Calif.) and sterile water. Each animal was anesthetized intramuscularly with tiletamine HCl plus zolazepam (1.4 mg/kg) (Telazol; Lederle Parenterals Inc, Carolina, Puerto Rico), xylazine (2.0 mg/kg) (X jet; Phoenix Scientific Inc, St. Joseph, Mo.), and atropine (0.04 mg/kg) (Atrojet SA; Phoenix Scientific Inc, St. Joseph, Mo.) followed by mask inhalation of an isoflurane (Isothesia; Abbott Laboratories, Chicago, Ill.) and oxygen combination. Approximately one hundred and forty (140) rectangular wounds measuring 10 mm×7 mm×0.5 mm were made in the paravertebral and thoracic area with a specialized electrokeratome fitted with a 7 mm blade. The wounds were separated from one another by 15 mm of unwounded skin.

### Example 21

### Treatments

Wounds were randomly assigned to each of six treatment groups. The treatment groups were A, SP Sepharose in a proprietary carbomer vehicle containing 2 mM Ca²⁺; B, the carbomer vehicle alone; C, Xeroform petrolatum gauze (Tyco Kendall, Seneca, S.C.); D, IntraSite hydrogel (Smith & Nephew, Largo, Fla.); E, Tielle polyurethane foam (Johnson & Johnson, New Brunswick, N.J.); and F, untreated air-exposed control. There was a total of n=30 wounds per treatment group/day, except for untreated air exposed where n was 70 for each day. The hydrogels were applied over the wounds and surrounding normal skin with a sterile tongue depressor to the approximate thickness of 1 mm. The wound dressing materials in groups A, B, and C were then covered with a Tegaderm polyurethane dressing (3M, St. Paul, Minn.) to prevent desiccation. On day 1 after treatment, the animals were anesthetized and the dressings observed to make sure they were still intact. All materials were kept in place until wound evaluation unless it was observed that the materials needed to be replaced. In order to assess the wounds, a portion of the dressing material was removed to uncover five wounds for evaluation.

### Example 22

### Assessment of Re-Epithelialization

Beginning on day 4 (after wounding on day 0), and on each day thereafter until all wounds were completely epithelialized, five wounds and the surrounding normal skin from each treatment group were excised from a pig using an electrokeratome with a 22 mm blade set at a depth of 0.7 mm. All specimens that were not excised intact were discarded. The excised skin containing the wound site was incubated in 0.5 M sodium bromide at 37° C. for 24 hours, allowing for a separation of the dermis from the epidermis. After separation, the epidermal sheet was examined macroscopically for defects. Defects were defined as holes in the epidermal sheet or as a lack of epidermal continuity in the area of the wound. Epithelialization was considered complete (healed) if no defects were present; any defect in the wound area indicated that healing was incomplete. For each treatment group, on each day the number of wounds healed (completely epithelialized) was divided by the total number of wounds in that group sampled on that day, and multiplied by 100. Statistical analysis was done with chi square with fourfold tables.

A hydrogel formulation containing SP agarose and 2 mM Ca²⁺ in a carbomer vehicle (henceforth referred to as ‘agarose in carbomer’) was tested on porcine dermal wounds. The carbomer vehicle had the appearance of a clear, thick viscous liquid. As controls, we tested a variety of other treatments. The hydrogel treatments remained in place and were readily absorbed by the skin. The Xeroform gauze treatment did not remain in place throughout much of the study and needed to be changed frequently. The Tielle foam caused slight re-wounding during the early assessment days. None of the wounds from any of the treatment groups showed any erythema or clinical signs of infection.

On day four, wounds treated with agarose in carbomer showed the highest percentage of complete epithelialization (73%) (Table 3). This was followed by the Xeroform gauze (60%) IntraSite hydrogel (43%), and Tielle foam (20%). None of the untreated air exposed wounds epithelialized on this day. Statistical significance was observed between all groups and the untreated group (p<0.001), between agarose in carbomer vs. carbomer (p<0.05), IntraSite hydrogel (p<0.05), or Tielle foam (p<0.001); and between Xeroform gauze vs. Tielle foam (p<0.01).

On day five, wounds treated with agarose in carbomer or carbomer vehicle were all completely epithelialized (Table 3). Those treated with Xeroform gauze and IntraSite hydrogel were close behind (97% and 93%). Wounds treated with the Tielle foam and untreated air exposed wounds were 83% and 13% completely epithelialized. Statistical significance was observed only between all treatment groups and the untreated control group (p<0.001).

On the sixth day, there was 100% complete epithealization of wounds except for those treated with the Tielle foam (97% epithealization) and the untreated wounds (54% epithealization). All groups showed significant differences compared to the untreated group (p<0.001); otherwise there were no inter-group differences. On the seventh day, all wounds that received treatment were 100% completely epithealized. The untreated wounds were 80% epithealized, and all treated groups showed significant differences compared to the untreated group (p<0.01). On day eight all wounds including the untreated wounds were completely epithealized, and as a result there were no statistically significant differences.

### Example 23

### Histology and Immunohistochemistry

Full thickness 8 mm biopsies were obtained through the center of the wounds. Skin sections were embedded in OCT (VWR, West Chester, Pa.), frozen on dry ice and then stored at −80° C. 10 μm cryosections were mounted on Superfrost Plus microscope slides (VWR). Sections were fixed in acetone for 10 minutes at room temperature, and then air-dried for 15 minutes. Slides were then rehydrated in water for 5 minutes. Sections were covered in Gill's #3 Accustain Hematoxylin Solution (Sigma-Aldrich) diluted 1:1 in water for 1 minute and were then rinsed with water for 3 minutes. Slides were dehydrated in 70% ethanol for 3 minutes, then 95% ethanol for 5 minutes. Sections were covered with 0.1% Eosin Y (Fisher Scientific, Pittsburgh, Pa.) in water for 1 minute. After rising off the Eosin Y, the slide was dehydrated in 100% ethanol for 5 minutes, then xylene for 10 minutes, and mounted with Permount (Fisher), as described previously.

Hematoxylin and eosin-stained cryosections of porcine skin from an unwounded region showed a normal epidermis (dark purple) with the presence of rete ridges (FIG. 15A). On the top surface, a thin stratum corneum was noted (bright mauve). As expected, the dermis has a normal basket weave collagen pattern. A section of an untreated day 4 wound (FIG. 15B) showed significant crust formation (FIG. 15B) separated in areas by an air gap over the dermis, with very little epidermis. A section of a wound at day 4 treated with agarose in carbomer (FIG. 15C) showed a mature epidermis covering the entire dermis. At day 7, an untreated wound (FIG. 15D) showed significant crust formation over the wound, with some epidermis migrating from a hair follicle (left side) (arrow, FIG. 15D). At day 7, a wound treated with agarose in carbomer (FIG. 15E) showed a mature epidermis over the wounded area. Together, the histology observations and percent epithelialization results indicate that treating porcine skin wounds with agarose in carbomer enhances epidermis formation sooner than control wounds.

TABLE 3

| | Complete epithelialization | | | | |
|---|---|---|---|---|---|
| Treatment | Day 4 | Day 5 | Day 6 | Day 7 | Day 8 |
| Untreated | 0/70 | 9/70 | 38/70 | 56/70 | 70/70 |
| | 0% | 13% | 54% | 80% | 100% |
| Agarose in | 22/30 | 30/30 | 30/30 | 30/30 | 30/30 |
| carbomer | 73% | 100% | 100% | 100% | 100% |
| carbomer | 10/30 | 30/30 | 30/30 | 30/30 | 30/30 |
| | 33% | 100% | 100% | 100% | 100% |
| Xeroform Gauze | 18/30 | 29/30 | 30/30 | 30/30 | 30/30 |
| | 60% | 97% | 100% | 100% | 100% |
| IntraSite | 13/30 | 28/30 | 30/30 | 30/30 | 30/30 |
| hydrogel | 43% | 93% | 100% | 100% | 100% |
| Tielle foam | 6/30 | 25/30 | 29/30 | 30/30 | 30/30 |
| | 20% | 83% | 97% | 100% | 100% |

Epithelialization of partial thickness porcine skin wounds. Wounds were treated with the indicated dressings. On days 4 through 8 after wounding, 70 untreated wounds and 30 treated wounds per dressing type were excised and the number of epithealized wounds was determined. The percentage of completely epithealized wounds was then determined.

To detect cytokeratin and collagen-I, slides were fixed in acetone for 10 minutes, followed by a 60 minute incubation of the slide in 4% BSA in PBS to block nonspecific binding. Slides were then incubated for 60 minutes in PBS with 4% BSA containing either 5 μg/ml anti cytokeratin monoclonal antibody (clone C-11, mouse IgGI, Sigma), or rabbit polyclonal anti-collagen-I antibodies (600-401-104, Rockland Inc, Gilbertsville, Pa.). Irrelevant mouse IgG 1 monoclonal antibodies (BD Biosciences, San Jose, Calif.) or irrelevant rabbit polyclonal antibodies (Jackson ImmunoResearch, West Grove, Pa.) at 5 μg/ml were used as controls. Slides were then washed in six changes of PBS over thirty minutes. The slides were then incubated with either 2.5 μg/ml biotinylated rat F(ab′)2 anti-mouse IgG (Jackson ImmunoResearch,) or biotinylated goat F(ab′)2 anti-rabbit IgG (Southern Biotechnology, Birmingham, Ala.) with 4% BSA in PBS. After washing, the biotinylated antibodies were detected with a 1/200 dilution of ExtrAvidin alkaline phosphatase (Sigma) in PBS containing 4% BSA. Staining was developed with the Vector Red Alkaline Phosphatase kit (Vector Laboratories, Burlingame, Calif.) for 5 minutes and slides were then counterstained for ten seconds with Gill's #3 Hematoxylin Solution (Sigma) diluted 1:5 in water for ten seconds. The slides were rinsed in water and were then dehydrated and mounted as above.

Cytokeratin is a marker of epithelial tissue. A section of unwounded porcine skin showed staining with anti-cytokeratin antibodies in a layer between the epidermis and dermis (FIG. 16A). Similar staining was observed at day 10 for wounds treated with agarose in carbomer (FIG. 16C) and wounds treated with IntraSite hydrogel (FIG. 16D). This indicated that after healing with these two treatments, the skin formed a layer of cytokeratin-positive cells. However, at day 10, an untreated wound showed little expression of cytokeratin in the epithelial tissue, indicating that the untreated wounds were less mature than the treated wounds. The expression of cytokeratin around hair follicles in the untreated wounds served as an internal control of cytokeratin staining (arrow, FIG. 16B). We also stained sections with anti-collagen I antibodies as a marker for dermal integrity and remodeling. Normal porcine skin, and untreated, agarose in carbomer-treated, and IntraSite hydrogel-treated wounds at day 10 (FIGS. 16E-H) all showed staining with anti-collagen I antibodies in the dermis. Together, the data suggest that at day 10, agarose in carbomer and IntraSite hydrogel treatments do not appear to affect the amount of collagen in the dermis, but increase the amount of epithelial cytokeratin staining compared to untreated wounds.

The present inventors have observed that several agarose samples bind SAP (Examples 17-23). An agarose was chosen that showed high binding. It was found that in vitro this material can bind 2.1 μg SAP/mg wet weight agarose. Human serum contains 5-60 μg/ml SAP, with an average of ˜30 μg/ml. If it is assumed that the wound fluid after blood clotting contains a similar concentration of SAP, then those in vitro binding observations would indicate that placing 200 μg wet weight of agarose in a buffer containing 2 mM calcium on a wound containing 1 ml of wound fluid would reduce the SAP concentration in the wound fluid from ˜30 μg/ml to ˜0.02 μg/ml (FIGS. 13A and 13B). SAP completely inhibits fibrocyte differentiation in vitro at a concentration of 30 μg/ml. The EC50 for SAP inhibition of fibrocyte differentiation is ˜0.1 μg/ml. At 0.02 μg/ml there is no significant inhibitory effect of SAP on fibrocyte differentiation. This would then suggest that placing the same 200 μg wet weight of agarose in a buffer containing calcium on a wound containing 1 ml of wound fluid would reduce the SAP concentration from one which inhibits fibrocyte differentiation to a SAP concentration which permits fibrocyte differentiation. Accordingly, without being limited to any particular mechanism of action, the effects of the wound dressing on rate of wound epithelialization may be due to its effect on fibrocyte differentiation.

The primary sequences and molecular masses of SAP are highly conserved across species, and in the presence of millimolar concentrations of Ca²⁺, high EEO agarose binds SAP from a variety of species including human, mouse, cow, fish, toad, and pig. It is disclosed herein that in the presence of Ca²⁺, SP agarose binds a protein that we identified as porcine SAP based on its molecular mass on SDS-polyacrylamide gels, and cross-reactivity with anti-human SAP antibodies. According to the manufacturer, the anti-human SAP antibody used is an affinity-purified rabbit monoclonal antibody against a domain in the N terminal region of human SAP. In the N-terminal 100 amino acids, porcine SAP has 81% identity and 94% similarity to human SAP, supporting the idea that an anti-human SAP antibody will cross-react with porcine SAP, and thus that the 27 kDa protein in porcine serum that binds to SP agarose in the presence of Ca²⁺ is porcine SAP.

Immediately after blood clotting in a wound, the wound fluid is by definition mostly blood serum, and will thus contain SAP. During normal wound healing, the concentration of SAP in the wound fluid may decrease with time, due for example to some combination of degradation and ingestion by cells based on the opsonization of cell debris by SAP. Since SAP inhibits fibrocyte differentiation, and fibrocytes are observed in healing wounds, it may be that at some point in normal wound healing, the free SAP concentration falls below the point at which it inhibits fibrocyte differentiation, allowing fibrocytes to participate in wound healing.

It has been observed herein that for the partial thickness porcine skin wounds, the agarose in carbomer dressings caused faster wound healing than a variety of other treatments. Although the agarose in carbomer dressings could cause faster wound healing by an unknown mechanism, a reasonable explanation is that the agarose in the formulation was binding porcine SAP in the wound fluid, causing a rapid decrease in the free SAP concentration in the wound fluid and possibly in the upper cell layers of the wound. Compared to a normal wound, the rapid removal of free SAP would cause an equally rapid removal of the fibrocyte-inhibiting effect of SAP, allowing fibrocyte differentiation to occur earlier in the process of wound healing, thus speeding wound healing.

Fibrocytes participate in fibrotic lesions as well as wound healing. A systemic depletion of SAP thus might be deleterious. As disclosed herein, it has been observed that the agarose in carbomer dressings caused faster healing only of the wounds to which they were applied, without speeding healing of the other wounds on the same pig. This result is consistent with the agarose in carbomer dressings causing a local but not a systemic depletion of SAP. In humans, the average serum SAP concentration is ˜30 mg/liter, with a range of 5-60 mg/liter. If a wound had 10 grams wet weight of SP agarose placed on it, our observed Bmax of 2.1 mg of SAP bound/g agarose would predict a maximum of 21 mg SAP bound by the 10 g of agarose. Assuming 5 liters of blood volume in an adult, and thus a total of 150 mg SAP, depleting 21 mg of SAP would reduce the total circulating SAP concentration by a maximum of 14%. At a free SAP concentration of 30 mg/liter (30 μg/ml), SP agarose binds less SAP than the Bmax (FIG. 13A), so the actual amount of SAP depleted by 10 grams of agarose in an adult would be lower than 14% of the circulating SAP. Even a 14% depletion would reduce the serum SAP concentration to ˜25 mg/liter, well within the normal human range, so a 10 g agarose dressing should be safe to use on humans.

The ability of the agarose in carbomer dressings to speed wound healing in pigs suggests that these dressings might speed healing of human wounds. Fibrocytes are found in hypertrophic scars, and the ability to regulate fibrocyte differentiation by removal or addition of SAP might thus also allow reduction in the formation of hypertrophic scars. Consistent with this hypothesis, local and systemic SAP injections slow wound healing in mice.

Although only exemplary embodiments of the disclosure are specifically described above, it will be appreciated that modifications and variations of these examples are possible without departing from the spirit and intended scope of the disclosure.

What is claimed is:

1. A method of promoting wound healing in a mammal having a skin injury or laceration comprising:
supplying a wound dressing composition to a mammal having a skin injury or laceration, the skin injury or laceration containing Serum Amyloid P (SAP), in an amount and for a length of time sufficient to suppress the ability of the SAP to suppress monocyte differentiation into fibrocytes in the skin injury or lateration.
wherein the wound dressing composition comprises:
a Serum Amyloid P (SAP)-binding agarose, the SAP-binding agarose operable to promote healing of the skin injury or laceration in the mammal; and
a divalent cation in an amount sufficient to promote healing of the skin injury or laceration in conjunction with the SAP-binding agarose in a concentration sufficient to promote healing of the skin injury or laceration more quickly than the SAP-binding agarose alone.

2. The method of claim 1, further comprising increasing the number of fibrocytes present in the skin injury or laceration.

3. The method of claim 1, further comprising depleting SAP or suppressing SAP activity in the skin injury or laceration.

4. The method of claim 1, wherein the mammal is a primate.

5. The method of claim 1, further comprising supplying to the mammal having a skin injury or laceration an additional wound healing factor.

6. The method of claim 5, wherein the additional wound healing factor is selected from the group consisting of: interleukin (IL)-4, IL-13, fibroblast growth factor (FGF), transforming growth factor beta (TGFβ), and any combinations thereof.

7. The method of claim 5, wherein the additional wound healing factor comprises IL-13 supplied at a concentration of 0.1 to 10 ng/ml.

8. The method of claim 5, wherein the additional wound healing factor comprises IL-4 supplied at a concentration of 0.1 to 10 ng/ml.

9. The method of claim 5, wherein the cation comprises Ca²⁺.

10. The method of claim 1, wherein the SAP-binding agarose comprises high EEO agarose comprising a pyruvate acetyl of galactose.

11. The method of claim 1, wherein the SAP-bind agarose comprises a phosphoethanolamine moiety.

12. A wound dressing comprising:
a Serum Amyloid P (SAP)-binding agarose, the SAP-binding agarose operable to promote healing of a skin injury or laceration in a mammal; and
a divalent cation in a concentration of at least 0.3 mM.

13. The wound dressing of claim 12, further comprising an additional wound healing factor.

14. The wound dressing of claim 13, wherein the additional wound healing factor is selected from the group consisting of: interleukin (IL)-4, IL-13, fibroblast growth factor (FGF), transforming growth factor beta (TGFβ), and any combinations thereof.

15. The wound dressing of claim 13, further comprising IL-13 at a concentration of 0.1 to 10 ng/ml.

16. The wound dressing of claim 13, further comprising IL-4 at a concentration of 0.1 to 10 ng/ml.

17. The wound dressing of claim 12, wherein the cation comprises Ca²⁺.

18. The wound dressing of claim 12, wherein the SAP-binding agarose comprises high electroendosmosis (EEO) agarose.

19. The wound dressing of claim 18, further comprising approximately 1% (w/v) high EEO agarose.

20. The wound dressing of claim 18, wherein the high EEO agarose comprises a pyruvate acetal of galactose.

21. The wound dressing of claim 12, where in the SAP-binding agarose comprises a phosphoethanolamine moiety.

22. The wound dressing of claim 12, further comprising a bandage.
